(19) United States
(12) Patent Application Publication
(10) Pub. No.: US 2013/0095047 A1
(43) Pub. Date: Apr. 18, 2013

(54) COSMETIC COMPOSITION

(71) Applicants: Thomas Charles Castle, Chester (GB); Yasmine El Mourabit, Hillington (GB); David Alan Pears, Poynton (GB)

(72) Inventors: Thomas Charles Castle, Chester (GB); Yasmine El Mourabit, Hillington (GB); David Alan Pears, Poynton (GB)

(73) Assignee: REVOLYMER LIMITED, London (GB)

(21) Appl. No.: 13/511,910

(22) PCT Filed: Nov. 29, 2010
(86) PCT No.: PCT/GB2010/002193

§ 371 Date: Dec. 21, 2012

(30) Foreign Application Priority Data
Nov. 27, 2009 (GB) 0920879.4

(51) Int. Cl. A61K 8/91; A61Q 1/00; A61Q 1/06; A61Q 1/04

(52) U.S. Cl. CPC A61K 8/91; A61Q 1/04; A61Q 1/00; A61Q 1/06
USPC 424/59; 514/772.1; 424/63; 424/64

(57) ABSTRACT

A cosmetic composition including: (i) at least one amphiphilic copolymer; and (ii) one or more cosmetically acceptable diluents, excipients or carriers; wherein the amphiphilic copolymer is selected from the group consisting of a graft copolymer including a hydrophobic straight or branched chain carbon-carbon backbone having at least one hydrophilic side chain attached thereto; a graft copolymer comprising a hydrophilic straight or branched chain backbone having at least one hydrophobic side chain attached thereto; a block copolymer including at least one hydrophilic block and at least one hydrophobic block in a straight or branched chain backbone; and a cross-linked/network copolymer.

The present invention relates to a cosmetic composition comprising an amphiphilic copolymer and a process for the preparation thereof.

### BACKGROUND TO THE INVENTION

Consumers increasingly demand multifunctional performance personal products. This particularly applies to cosmetic products where it is frequently desired that the products possess several different properties. For instance, there is a demand for lipstick that is longer lasting on the lips and has good moisturisation and skin feel, whilst retaining a high gloss and excellent cosmetic appearance. Typically to achieve these effects, a number of different components are added to the formulations. However, for reasons of manufacturing economy, it is desirable to reduce the number of components to the minimum. It is also frequently difficult to combine all the desired components due to formulation incompatibilities. Cosmetic or make-up products may be further defined as lip, face or eye make-up products.

Broadly speaking, products intended to be used on the lips fall into two categories: those that are used primarily to change the cosmetic appearance of the lip, i.e. make-up products; and those intended to ensure the health of the lips and lip tissue.

The concept of applying cosmetics to lips to enhance their appearance has been known for many years. For example, it is believed that the ancient Egyptians wore a simple form of lipstick possibly derived from insect sources (see, for example, Chemical & Engineering News, Jul. 12, 1999, Volume 77, Number 28, p. 31). Lip make-up has been in frequent use since then and has grown to be a major consumer product over more recent years. Amongst the most typical lip make-up products are lipstick, lip gloss, lip liner, lip plumper, lip balm, lip sheers, lip ink, lip conditioner, lip primer, and lip boosters.

Historically, the most important lip cosmetic is lipstick, a solid stick product typically used to enhance the colour of lips usually fashioned as a cylindrical product of varying diameter dispensed from a specially designed tube. Many different products are commercially available and claim to have a variety of different effects, e.g. providing enhanced gloss, moisturisation, longer lasting formulations and/or transfer resistant films. Lip gloss is a viscous liquid product that is primarily used to increase the gloss of lips, as well as potentially deposit colour. Consequently, the amount of pigment in a lip gloss formulation is comparatively small, with some formulations containing just a pearlescent pigment to give shine to the lips. Lip liner is usually sold in pencil format and is applied after the application of lipstick to fill uneven areas on the outer edges of the lips to give a more uniform shape. Lip stains are prepared with a dye to provide colour on the lips. Products are typically sold in small bottles and are applied either by an applicator or by finger. Products are typically formulated to provide waterproof long lasting colour. Lip plumpers are formulated to give lips a fuller appearance. Typically, this is achieved by the use of an active ingredient that causes irritation of the lip surface resulting in fuller, or pouty, lips. Up balm is sometimes classed as a cosmetic and is used to treat chapped or dry lips, angular cheilitis or stomatitis, and cold sores. Many lip products also incorporate extra functionality such as sun protection.

Facial cosmetics may further divided into a range of products. Typical forms of face make-up include foundation, face powder, concealer, blusher and bronzer. Foundation is added to the face to create a uniform complexion or possibly alter the colour of the skin. Face powder is sometimes used to set foundation on the face and can also be reapplied throughout the day to control oil. Concealer differs from foundation in that it is intended to hide a range of localised imperfections, including spots, pimples, and scars, rather than the cover the whole face. Blusher (also called Blush or rouge) is a red or similar coloured cosmetic applied to the cheeks to add a youthful or healthy appearance and emphasise cheekbones. Bronzer is applied to add a bronze or golden tone not dissimilar to that expected from exposure to UV light.

Eye cosmetics are typically is applied in the form of eye shadow, eyeliner or mascara. Eye shadow is typically applied to the areas around the eyes, on the eyelids, and under the eyebrows, with the intention of making the eyes more attractive or stand-out. Eyeliner is applied to define the contours around the eye. Mascara is applied to the eye lashes to change the colour, shade, volume or length or eye lashes. Cosmetics are also applied to the eye brow to change their colour and define them.

The present invention seeks to provide a multifunctional agent for use in cosmetic compositions, more specifically face make-up, eye make-up, and lip make-up and care products. More specifically, but not exclusively, the invention seeks to provide cosmetic compositions (for instance lipsticks, lip balms and foundation) that exhibit one or more of increased adhesion to the lips during normal wear, an enhanced feeling of moisturisation, and/or enhanced organoleptic (i.e. skin feel) properties.

### STATEMENT OF INVENTION

The present invention relates to a cosmetic composition comprising:

(i) at least one amphiphilic copolymer, and
(ii) one or more cosmetically acceptable diluents, excipients or carriers;
wherein the amphiphilic copolymer is selected from the group consisting of a graft copolymer comprising a hydrophobic straight or branched chain carbon-carbon backbone having at least one hydrophilic side chain attached thereto; a graft copolymer comprising a hydrophilic straight or branched chain backbone having at least one hydrophobic side chain attached thereto; a block copolymer comprising at least one hydrophilic block and at least one hydrophobic block in a straight or branched chain backbone; and a cross-linked/network copolymer.

Advantageously, cosmetic compositions according to the invention exhibit increased adhesion during normal wear, making them longer lasting. In addition, the cosmetic compositions of the invention provide an enhanced feeling of moisturisation, and enhanced organoleptic (i.e. skin feel) properties.

A second aspect of the invention relates to a process for preparing a cosmetic composition, of the type mentioned above said process comprising melting an amphiphilic copolymer with one or more cosmetically acceptable diluents, excipients or carriers to form a homogenous product.

### DESCRIPTION OF FIGURES

FIG. 1: Comparison of Organoleptic Properties of Lipstick Formulation with and without PG1

FIG. 2: Comparison of Preference of Lipstick Formulation with PG1 vs. without PG1

FIG. 3: Comparison of Organoleptic Properties of Lip Balm Formulation with and without PG1

FIG. 4: Comparison of Preference of Lip Balm Formulation with PG1 vs. without PG1

FIG. 5: Percentage of Individuals Whom Noted the Formulation was Longer Lasting

FIG. 6: Comparison of the Organoleptic Properties of Various Lipstick Formulations

### DETAILED DESCRIPTION OF THE INVENTION

As used herein, the term “copolymer” refers to a polymeric system in which two or more different monomers are polymerised together.

As used herein, the “term” amphiphilic copolymer refers to a copolymer in which there are clearly definable hydrophilic and hydrophobic portions.

As used herein, the term “alkyl” encompasses a linear or branched alkyl group of about 1 to about 20 carbon atoms, preferably about 1 to about 10 carbon atoms, more preferably about 1 to about 5 carbon atoms. For example, a methyl group, an ethyl group, an isopropyl group, a n-propyl group, a butyl group, a tert-butyl group or a pentyl group.

As used herein, the term “aryl” encompasses any functional group or substituent derived from an aromatic ring or a heteroaromatic ring, preferably a C6 to C20 aromatic ring, for example, phenyl, benzyl, tolyl or napthyl.

The cosmetic composition of the present invention comprises at least one amphiphilic copolymer. In one embodiment, the composition of the present invention comprises between about 1 and about 4 amphiphilic copolymers, for example 1, 2, 3, or 4 copolymers, preferably one or two copolymers, most preferably one copolymer.

In any embodiment of the present invention, the amphiphilic copolymer may have a hydrophilic-lyphophilic (or hydrophobic) balance (HLB) as measured by Griffin's method of less than or equal to about 15, preferably less than or equal to about 10, more preferably between about 1 and about 10, yet more preferably between about 2 and about 9, for example, between about 3 and about 8. The Griffin method values are calculated by: hydrophilic-lyphophilic balance=20×molecular mass of the hydrophilic portion/molecular mass of the whole molecule.

The molecular mass of the hydrophilic and hydrophobic portions of the polymer may be estimated from the quantities of the relevant monomers put in as feedstocks in the amphiphilic copolymer's manufacture and understanding of the kinetics of the reaction. The composition of the final product may be checked by comparing the relevant intensities of signals from each block or portion using ¹H nuclear magnetic resonance spectroscopy. Alternatively any other quantitative spectroscopic technique such as infra-red spectroscopy or ultra-violet visible spectroscopy may be used to confirm the structure, provided the different portions give clearly identifiable and measurable contributions to the resulting spectra. As described in Reference Method A gel permeation chromatography (GPC) may be used to measure the molecular weight of the resulting materials.

In one embodiment of the invention, the amphiphilic copolymer is a graft copolymer comprising a hydrophobic straight or branched chain carbon-carbon backbone having at least one hydrophilic side chain attached thereto.

In a preferred embodiment of the invention, the hydrophilic side chains of the graft copolymer are each independently of formula (I),

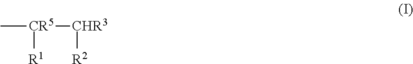

wherein R¹ and R² are each independently H, —C(O)WR⁴ or —C(O)Q;
provided that at least one of R¹ and R² is the group —C(O)Q;
or R¹ and R² together form a cyclic structure together with the carbon atoms to which they are attached, of formula (II)

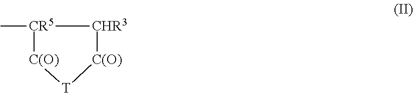

wherein:
R³ and R⁵ are each independently H or alkyl;

### W is O or NR⁴;

Q is a group of formula —X¹—Y—X²P;
T is a group of formula —N—Y—X²—P;

### X¹ is O, S or NR⁴;

### X² is O, S, (CH₂)ₚ or NR⁴;

p is 0 to 6;
each R⁴ is independently H or alkyl;
P is H or another backbone; and
Y is a hydrophilic polymeric group.

In a preferred embodiment of the invention, the hydrophilic polymeric group Y is a poly(alkylene oxide), polyglycidol, poly(vinyl alcohol), poly(ethylene imine), poly(styrene sulphonate) or poly(acrylic acid). More preferably, the hydrophilic polymeric group Y is a poly(alkylene oxide), such as poly(ethylene oxide) or a copolymer thereof.

In a further preferred embodiment of the invention, the hydrophilic polymeric group Y is of formula -(Alk¹-O)_{b}-(Alk²-O)_{c}—, wherein Alk¹ and Alk² are each independently an alkylene group having from 2 to 4 carbon atoms, and b and c are each independently an integer from 1 to 125; provided that the sum b+c has a value in the range of from about 10 to about 250, more preferably, from about 10 to about 120.

In a further preferred embodiment of the invention, the graft copolymer has from 1 to 5000, preferably from about 1 to about 300, and more preferably from about 1 to about 150, pendant hydrophilic groups attached thereto. For example, the graft copolymer may have between about 1 to about 10, between about 1 to about 5, or between about 2 to about 8 pendant hydrophilic groups attached thereto.

Where the amphiphilic copolymer is a graft copolymer, each side chain of the graft polymer preferably has a molecular weight from about 800 to about 10,000. For example, each side chain may have a molecular weight between about 1000 to about 7500, between about 2500 to about 5000 or between about 6000 and about 9000.

A graft copolymer is typically produced by the reaction of hydrophilic grafts with a single reactive site on the carbon-carbon backbone, i.e. the reaction uses monofunctional grafts. In order to create a cross-linked or chain extended copolymer it is necessary to incorporate a hydrophilic graft that has two sites that will react with the carbon-carbon backbone; i.e. a difunctional hydrophilic graft that can act as a cross-linking agent is used.

Preferably, the cross-linked or chain extended copolymers comprise a linear or branched carbon-carbon backbone and a difunctional graft or a mixture of monofunctional and difunctional grafts. More preferably, the cross-linked or chain extended copolymers comprise a carbon-carbon backbone functionalized with maleic anhydride or a derivative thereof (as described herein) and an alkylene oxide such as those described in formula (II). Most preferably, the cross-linked or chain extended copolymers comprise a carbon-carbon backbone derived from polyisoprene or polybutadiene functionalized with maleic anhydride or a derivative thereof, and further comprise hydrophilic grafts being polyethylene oxide or a copolymer thereof.

In one preferred embodiment of the invention, the carbon-carbon polymer backbone is derived from a homopolymer of an ethylenically-unsaturated polymerizable hydrocarbon monomer or from a copolymer of two or more ethylenically-unsaturated polymerizable hydrocarbon monomers.

More preferably, the carbon-carbon polymer backbone is derived from an ethylenically-unsaturated polymerizable hydrocarbon monomer containing 4 or 5 carbon atoms.

In one highly preferred embodiment of the invention, the carbon-carbon polymer backbone is derived from isobutylene, 1,3-butadiene, isoprene or octadecene, or a mixture thereof.

In one preferred embodiment of the invention, the copolymer comprises a carbon-carbon backbone (e.g. polyisoprene or polybutadiene) onto which maleic anhydride or maleic anhydride acid/ester groups have been grafted. Preferably, the carbon-carbon backbone comprises from about 1 to about 50 mol % maleic anhydride group. As used herein, the term maleic anhydride (MA) group encompasses maleic anhydride, maleic acid and salts thereof and maleic acid ester and salts thereof and mixtures thereof.

The maleic anhydride group coupling chemistry provides a convenient method for attaching the grafts to the copolymer backbone. However, the skilled person would appreciate that other functional groups would be equally effective in this regard.

By way of example, the reaction of another acyl group (e.g. a suitable carboxylic acid or acyl chloride) with a hydroxyl functionalised polymer will be suitable for forming an ester linkage between the graft and backbone. Various strategies for performing coupling reactions, or click chemistry, are also known in the art and may be utilised by functionalising the backbone with suitable groups, possibly in the presence of a suitable catalyst. For instance the reaction of an alkyl or aryl chloride group on the backbone with a hydroxyl group for instance (i.e. a Williamson coupling), or the reaction of a silicon hydride with an allyl group (a hydrosilyation reaction) could be utilised.

Preferably, the carbon-carbon backbone comprises from about 1 to about 50 mol % maleic anhydride.

In one preferred embodiment, the backbone of the amphiphilic polymer has a molecular weight from about 1,000 to about 10,000.

In one preferred embodiment of the invention, the carbon-carbon backbone is a copolymer of:

(i) maleic anhydride, maleic acid or salts thereof or maleic acid ester or salts thereof or a mixture thereof; and
(ii) one or more ethylenically-unsaturated polymerizable monomers.

The MA group monomer is thus present in the actual backbone rather than pendant to it.

A number of such materials are available commercially, most typicaly obtained by the radical polymerisation of a mixture of a maleic anhydride group and one or more other ethylenically unsaturated monomers. It will be envisioned that any number of monomers, though most typically a mixture of a maleic anhydride group and one other monomer (to make a bipolymer) or two other polymers (to make a terpolymer) will be used.

Preferably, the maleic anhydride group monomer is maleic anhydride.

Preferably, the other monomer is ethylene, isobutylene, 1,3-butadiene, isoprene, a C10-C20 terminal alkene, such as octadecene, styrene, or a mixture thereof. Most preferably, the other monomer is isobutylene or octadecene.

The percentage of the monomers, and thus functionality in the resulting polymer, may be altered to provide optimal fit to the application. One advantage of backbones prepared by such a method is that they offer the potential for higher loadings of maleic anhydride potentially available for reaction with hydroxy, amine, or sufide functionalised grafts (e.g. suitable PEOs, MPEOs or amine functionalised alkyl ethxoylates like certain Jeffamines).

In one aspect of the invention the backbone is an alternating copolymer prepared by mixing and susbsequently polymerising equimolar quantities of a MA group and another monomer.

A particularly preffered backbone copolymer is polyisobutylene-alt-maleic anhydride) (PIB-alt-MA):

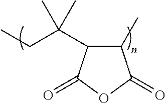

wherein n is between 5 and 4000, more preferably 10 and 1200.

This polymer is available commercialy from Sigma-Aldrich and Kuraray Co. Ltd; Kuraray supply the material under the trade name ISOBAM.

A further preffered backbone copolymer is poly(maleic anhydride-alt-1-octadecene) (C18-alt-MA) (available from the Chevron Philips Chemical Company LLC).

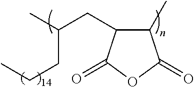

wherein n is between 5 and 500, more preferably 10 and 150.

Chevron Philips make a range of materials (both high and low viscosity) in their PA18 Polyanhydride resins range that are preffered backbones in the invention. PA18 is a solid linear polyanhydride resin derived from 1-octadecene and maleic anhydride in a 1:1 molar ratio.

It will be appreciated by those skilled in the art that a number of other backbones in which maleic anhydride is included in the backbone, either by grafting the maleic anhydride as an adduct, or by copolymerising maleic anhydride with one or more other monomers are useful in the invention.

A range of polybutadiene polymers functionalised with maleic anhydride are sold under the Ricon brand by Sartomer (e.g. Ricon 130MA8) and Lithene by Synthomer (e.g. N4-9000-10MA). A number of useful backbones are also manufactured by Kraton (e.g. Kraton FG) and Lyondell (e.g Plexar 1000 series) in which maleic anhydride is grafted onto polymers or copolymers of monomers such as ethylene, propylene, butylene, styrene and/or vinyl acetate.

Polystyrene-alt-maleic anhydride) is available from a number of suppliers including Sartomer under the SMA trade name. Poly(ethylene-alt-maleic anhydride) is available from a number of suppliers including Vertellus under the ZeMac trade name. Poly(methyl vinyl ether-alt-maleic anhydride) is available from International Speciality Products under the Gantrez trade name. Poly(ethylene-co-butyl acrylate-co-maleic anhydride) materials can be obtained from Arkema, and are sold under the trade name of Lotader (e.g. 2210, 3210, 4210, and 3410 grades). Copolymers in which the butyl acrylate is replaced by other alkyl acrylates (including methyl acrylate [grades 3430, 4404, and 4503] and ethyl acrylate [grades 6200, 8200, 3300, TX 8030, 7500, 5500, 4700, and 4720) are also available and also sold in the Lotader range. A number of the Orevac materials (grades 9309, 9314, 9307 Y, 9318, 9304, 9305) are suitable ethylene-vinyl acetate-maleic anhydride terpolymers.

In many cases in addition to, or instead of a maleic anhydride functionalised material a derivative a diacid, mono ester form, or salt is offered. As will be obvious to those skilled in the art many of these are also suitable in the invention.

Similarly, suitable side chains precursors are those discussed below, such as mono methoxy poly(ethylene oxide) (MPEO), poly(vinyl alcohol) and poly(acrylic acid). These may for instance be purchased from the Sigma-Aldrich company. Suitable polyethylene imines are available from BASF under the Lupasol trade name.

In one preferred embodiment, the amphiphilic copolymer is prepared by reacting a compound of formula (III),

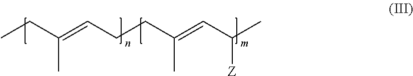

wherein Z is a group of the formula (IV),

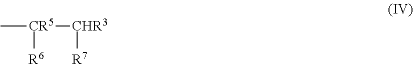

wherein R³ and R⁵ are each independently H or alkyl, and R⁵ and R¹ are each independently H or an acyl group, provided that at least one of R⁶ and R⁷ is an acyl group, or R⁸ and R⁷ are linked to form, together with the carbon atoms to which they are attached, a group of formula (V),

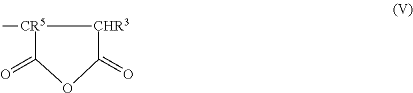

where n and m are each independently an integer from 1 to 20 000. Preferably m is 1 to 1000, more preferably 1 to 100 and yet more preferably 10 to 50. Preferably n is 1 to 5000, more preferably 5 to 2000 and yet more preferably 10 to 1000. Preferably,
m is 1 to 100 and n is 5 to 2000.
with a side chain precursor of formula (VI)

HX¹—Y—X²P (VI)

wherein:

### X¹ is O, S or NR⁴;

### X² is O, S, (CH₂)ₚ or NR⁴;

p is 0 to 6;
each R⁴ is independently H or alkyl;
P is H or another backbone; and
Y is a hydrophilic polymeric group.

In one preferred embodiment, the amphiphilic copolymer is prepared by reacting a compound of formula (IIIa),

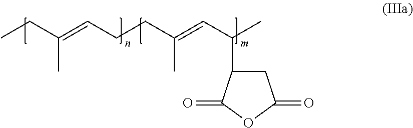

where n and m are as defined above, with a side chain precursor of formula (VI) as defined above.

In one preferred embodiment, the side chain precursor is of formula (VIa)

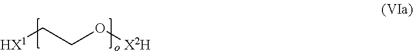

wherein X¹ is O or NH and X² is (CH₂)ₚ and o is an integer from 5 to 250, preferably 10 to 100.

In another preferred embodiment, the side chain precursor is of formula (VIb)

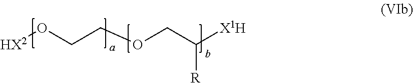

wherein R is H or alkyl, X¹ is O or NH and X² is (CH₂)ₚ and the sum of a and b is an integer from 5 to 600, preferably 10 to 100.

In one particularly preferred embodiment of the invention, the copolymer is prepared by grafting a monofunctional hydrophilic polymer such as poly(ethylene glycol)/poly(ethylene oxide) onto the maleic anhydride residues on the carbon-carbon backbone to form an amphiphilic copolymer of formula (VII),

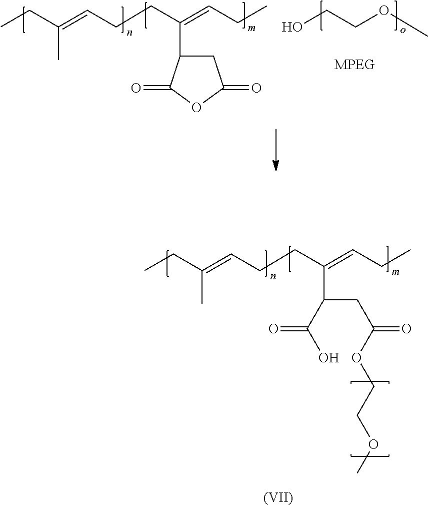

wherein each of m and n is independently an integer from 1 to 20 000. Preferably m is 1 to 1000, more preferably 1 to 100 and yet more preferably 10 to 50. Preferably n is 1 to 5000, more preferably 5 to 2000 and yet more preferably 10 to 1000. Preferably, m is 1 to 100 and n is 5 to 2000. Preferably o is an integer from 5 to 600, preferably 10 to 100.

The above example shows an alcohol functionalized PEO reacting with the maleic anhydride on a PIP-g-MA backbone. Suitable PIP-g-MA backbones are commercially available (for example, LIR-403 grade from Kuraray, which has approximately 3.5 MA units per chain).

Further details on functionalizing polyisoprene with maleic anhydride may be found in WO 06/016179, WO08/104,546, WO08/104,547, WO 09/68569 and WO 09/68570, the contents of which are herein incorporated by reference.

In one preferred embodiment, the copolymer is prepared by adding a ratio of 2:8 equivalents of MPEG with respect to each maleic anhydride (MA) group. This essentially enables complete conversion of the maleic anhydride groups into the PEG functionalized esters.

In another preferred embodiment, the copolymer is prepared by adding a 1:1 ratio of methoxy poly(ethylene oxide) (MPEO) to maleic anhydride. After complete reaction of the MPEO, another (second) (dihydroxy) poly(ethylene oxide) (PEO) of any molecular weight (e.g. 2000, 4000, 6000, 8000 and 10000) can be added. It will be understood by those skilled in the art that MPEO, poly(ethylene oxide) methyl ether, methoxy poly(ethylene glycol) (MPEG), and poly(ethylene glycol) methyl ether are alternative methods of naming the same structure. Similarly PEO is also sometimes referred to as poly(ethylene glycol) (PEG) in the art.

In addition to functionalising unreacted maleic anhydride units, it is also possible to graft PEG or another graft onto the corresponding diacid or a mono ester derivative of MA. This will result in new PEG ester links in the place of the COOH functionality. Two suitable backbones are illustrated below.

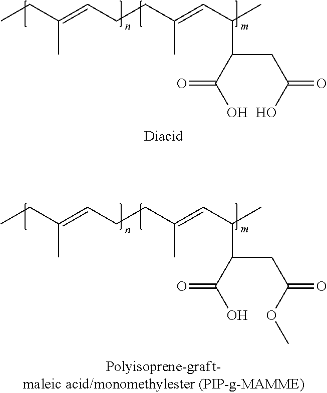

Thus, in one preferred embodiment, the amphiphilic copolymer is prepared by reacting a polymer precursor of formula (IIIb),

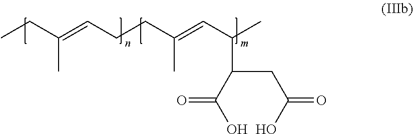

where n and m are as defined above, with a side chain precursor of formula (VI) as defined above.

In another preferred embodiment, the amphiphilic copolymer is prepared by reacting a polymer precursor of formula (IIIc),

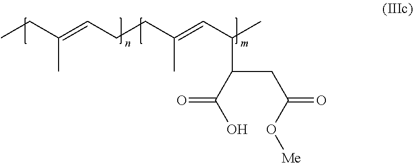

where n and m are as defined above, with a side chain precursor of formula (VI) as defined above.

In an alternative preferred embodiment, the copolymer of the invention is derived from —SH or nitrogen based (NH₂ or NHR) moieties.

In one particularly preferred embodiment, the copolymer comprises an NH₂ functionalized material. Preferably, for this embodiment, the amphiphilic copolymer is prepared from a side chain precursor of formula (VIc)

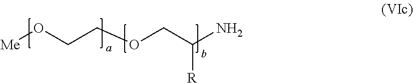

wherein R is H or alkyl, more preferably H or Me, and the sum of a and b is an integer from 5 to 250, preferably 10 to 100.

More preferably, the amphiphilic copolymer is of formulae (VIIIa) or (VIIIb) and is prepared by the following reaction:

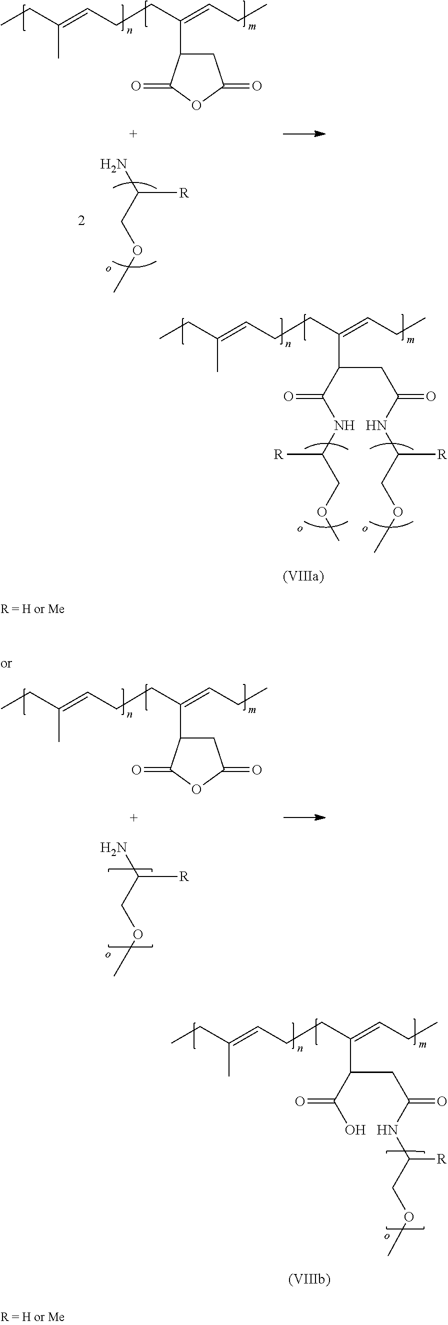

wherein each of m and n is independently an integer from 1 to 20 000. Preferably m is 1 to 1000, more preferably 1 to 100 and yet more preferably 10 to 50. Preferably n is 1 to 5000, more preferably 5 to 2000 and yet more preferably 10 to 1000. Preferably, m is 1 to 100 and n is 5 to 2000. Preferably o is an integer from 5 to 600, preferably 10 to 100.

The NH₂ functionalized material depicted above comprises two grafts on each MA, which is not possible with MPEO. This is due to the greater reactivity of the NH₂ groups compared with OH. In addition to grafting two chains per maleic anhydride unit, the greater reactivity of the NH₂ units with respect to OH leads to a product containing very small quantities of free graft.

In any of the above embodiments, the compounds of formula (III) may be replaced by compounds of formulae (IX) and (X):

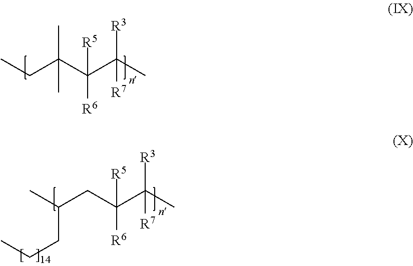

wherein n′ is 5 to 4000 and R³, R⁵, R⁶ and R⁷ are as previously defined.

Similarly, compounds of formulae (IIIa), (IIIb) and (IIIc) in any of the embodiments above may be replaced by compounds of formulae (IXa) or (Xa); (IXb) or (Xb); and (IXc) or (Xc), respectively:

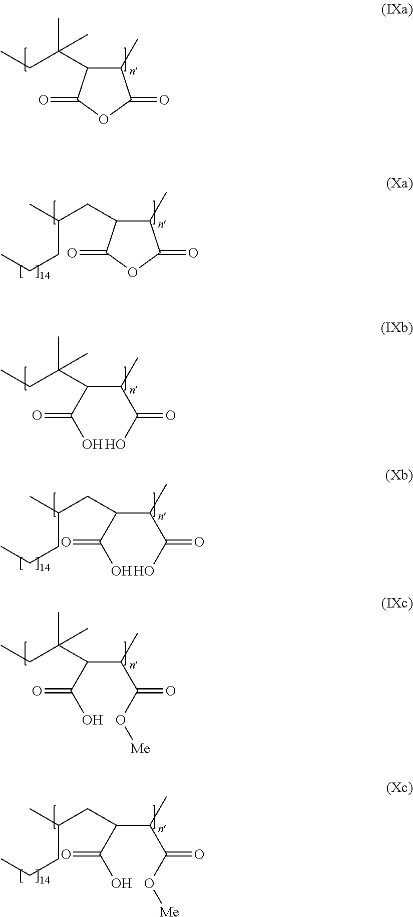

wherein n′ is as defined for compounds of formulae (IX) and (X).

In one preferred embodiment, the hydrophilic groups grafted onto the maleic anhydride groups are polymers of ethylene oxide (i.e. PEOs) copolymerised with propylene oxide. In this embodiment, the amount of propylene oxide is preferably between 1 and 95 mol percent of the copolymer, more preferably between 2 to 50 mol percent of the copolymer, and most preferably between 5 to 30 mol percent of the copolymer.

Preferably, the side chain precursor is of formula,

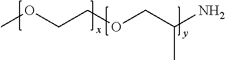

wherein x is 5 to 500, more preferably 10 to 100 and y is independently 1 to 125, more preferably 3 to 30. Preferably, x+y=6 to 600, more preferably 13 to 130. The distribution of ethylene and propylene oxide units may be in the form of blocks as depicted above or as a statistical mixture. In any case the molar ratio of ethylene oxide to propylene oxide in the copolymer will favour ethylene oxide. Such side chain precursors are sold commercially by Huntsman under the Jeffamine name.

Alternatively, it is possible to use a polymer that has two rather than one functional (e.g. OH, NH₂) units rather than one, in which both groups can react with the maleic anhydride. If these maleic anhydride groups are on different backbones, a cross-linked (or network) polymer can be formed. By controlling the ratio of graft to backbone, or by using mixtures with mono-functionalised materials, the degree of cross-linking can be controlled. Thus, it is possible to produce a material that resembles a chain extended graft copolymer (i.e. 2 or 3 graft copolymers) rather than a network by using a mixture of PEO and MPEO which chiefly comprises MPEO.

In one particularly preferred embodiment, the amphiphilic copolymer is prepared from a mixture of PIP-g-MA (polyisoprene with grafted maleic anhydride) together with MPEO (methoxy poly(ethylene oxide) and/or PEO poly(ethylene oxide). Preferably, the MPEO and PEO have a molecular weight of about 2000.

In one particularly preferred embodiment, the amphiphilic copolymer is prepared from a mixture of PIP-g-MaMme (polyisoprene with grafted maleic monoacid monoester) together with MPEO (methoxy poly(ethylene oxide)) and/or PEO (poly(ethylene oxide)). Preferably, the MPEO and PEO have a molecular weight of about 2000.

In an alternative embodiment of the invention, the amphiphilic copolymer is a graft copolymer comprising a hydrophilic straight or branched chain backbone having at least one hydrophobic side chain attached thereto.

In a preferred embodiment of the invention, the hydrophilic straight or branched chain backbone is a poly(alkylene oxide), polyglycidol, poly(vinyl alcohol), poly(ethylene imine), poly(styrene sulphonate) or poly(acrylic acid). More preferably, the hydrophilic straight or branched chain backbone is a poly(alkylene oxide) or copolymer thereof, such as poly(ethylene oxide), or a copolymer thereof.

In a preferred embodiment of the invention, the hydrophobic side chain is a hydrocarbon polymer, i.e. a side chain containing only carbon and hydrogen atoms.

More preferably, the hydrophobic side chain is derived from a homopolymer of an ethylenically-unsaturated polymerizable hydrocarbon monomer or from a copolymer of two or more ethylenically-unsaturated polymerizable hydrocarbon monomers.

In a highly preferred embodiment, the hydrophobic side chain may be derived from an ethylenically unsaturated hydrocarbon monomer having from about 2 to about 30 carbon atoms, preferably having from about 3 to about 20 carbon atoms, even more preferably having from about 4 to about 10 carbon atoms, most preferably having about 4 or about 5 carbon atoms, or a mixture thereof. For example, the hydrophobic side chain may be derived from ethylene, propylene, isobutylene, butadiene (1,3-butadiene), isoprene, a C10-C20 terminal alkene, such as octadecene, styrene, or a mixture thereof.

Most preferably, the hydrophobic side chain is polyisoprene, polybutadiene or a co-polymer thereof.

In a further preferred embodiment of the invention, the graft copolymer has from 1 to 5000, preferably from about 1 to about 300, and more preferably from about 1 to about 150, pendant hydrophobic groups attached thereto. For example, the graft copolymer may have between about 1 to about 10, between about 1 to about 5, or between about 2 to about 8 pendant hydrophobic groups attached thereto.

Where the amphiphilic copolymer is a graft copolymer, each side chain of the graft polymer preferably has a molecular weight from about 300 to about 10,000. For example, each side chain may have a molecular weight between about 300 to about 600, about 1000 to about 7500, between about 2500 to about 5000 or between about 6000 and about 9000.

The hydrophobic side chains may be attached to the hydrophilic backbone using the same methods of attachment employed to attach hydrophilic side chains to a hydrophobic backbone as described above.

A graft copolymer is typically produced by the reaction of hydrophobic grafts with a single reactive site on the hydrophilic backbone, i.e. the reaction uses monofunctional grafts. In order to create a cross-linked or chain extended copolymer it is necessary to incorporate a hydrophobic graft that has two sites that will react with the hydrophilicbackbone; i.e. a difunctional hydrophobic graft that can act as a cross-linking agent is used.

Preferably, the cross-linked or chain extended copolymers comprise a linear or branched hydrophilic backbone and a difunctional graft or a mixture of monofunctional and difunctional grafts. More preferably, the cross-linked or chain extended copolymers comprise a hydrophilic backbone which is poly(ethylene oxide), or a copolymer thereof functionalized with maleic anhydride or a derivative thereof, and further comprise hydrophobic grafts being polyisoprene, polybutadiene or a copolymer thereof.

In one preferred embodiment of the invention, the copolymer comprises a hydrophilic backbone, e.g. poly(ethylene oxide, onto which maleic anhydride or maleic anhydride acid/ester groups have been grafted. Preferably, the hydrophilic backbone comprises from about 1 to about 50 mol % maleic anhydride group. As used herein, the term maleic anhydride (MA) group encompasses maleic anhydride, maleic acid and salts thereof and maleic acid ester and salts thereof and mixtures thereof. For example, the copolymer may be prepared from a poly(ethylene oxide) backbone having maleic anhydride, acid or a salt or ester thereof grafts by reacting said backbone with an OH, NH2, NHR, or SH functionalized hydrophobic side chain.

The maleic anhydride group coupling chemistry provides a convenient method for attaching the grafts to the copolymer backbone. However, the skilled person would appreciate that other functional groups would be equally effective in this regard.

By way of example, the reaction of another acyl group (e.g. a suitable carboxylic acid or acyl chloride) with a hydroxyl functionalised polymer will be suitable for forming an ester linkage between the graft and backbone. Various strategies for performing coupling reactions, or click chemistry, are also known in the art and may be utilised by functionalising the backbone with suitable groups, possibly in the presence of a suitable catalyst. For instance the reaction of an alkyl or aryl chloride group on the backbone with a hydroxyl group for instance (i.e. a Williamson coupling), or the reaction of a silicon hydride with an allyl group (a hydrosilyation reaction) could be utilised.

Preferably, the hydrophilic backbone comprises from about 1 to about 50 mol % maleic anhydride.

In one preferred embodiment, the backbone of the amphiphilic polymer has a molecular weight from about 1,000 to about 100,000.

In one aspect of the invention the backbone is an alternating copolymer prepared by mixing and susbsequently polymerising equimolar quantities of a MA group and another monomer.

It will be appreciated by those skilled in the art that a number of other backbones in which maleic anhydride is included in the backbone, either by grafting the maleic anhydride as an adduct, or by copolymerising maleic anhydride with one or more other suitable monomers are useful in the invention. It will be understood by those skilled in the art that the monomers capable of undergoing copolymerisation with maleic anhydride, typically contain unsaturation, for instance acrylic acid.

In many cases in addition to, or instead of a maleic anhydride functionalised material a derivative a diacid, mono ester form, or salt is offered. As will be obvious to those skilled in the art many of these are also suitable in the invention.

In a further embodiment of the invention, the amphiphilic copolymer is a block copolymer comprising hydrophilic blocks and hydrophobic blocks in a straight or branched chain backbone.

In one embodiment of the invention, the straight or branched chain carbon-carbon backbone has at least one side chain attached thereto. The side chain(s) may be hydrophobic or hydrophilic. Examples of suitable side chains include those described above with reference to amphiphilic graft copolymers. Preferably the block copolymer has a straight chain backbone comprising hydrophilic blocks and hydrophobic blocks. In a further preferred embodiment, the amount of hydrophilic polymer by weight in the final composition is between from about 5 to about 60%.

Block copolymers may be synthesised by a number of routes including sequential polymerisation of two or more monomers using the same polymerisation technology, converting one polymerisation mechanism to another, or by coupling together two different polymeric blocks. A number of appropriate materials are available commercially and are suitable for use in the invention.

In one preferred embodiment the block copolymer has the structure:

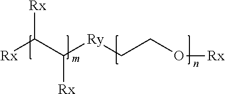

wherein Rx is an alkyl, aryl or H and Ry is O, NRₓ Si(Rx)₂.

Preferably the block copolymers are copolymers of ethylene oxide and an alkene, diene or polyene; preferably ethylene, propylene, isoprene or butadiene. In one preferred embodiment the block copolymer has the structure:

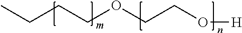

wherein m is 3 to 100, most preferably 10 to 30 and n is independently 3 to 100, most preferably 4 to 40.

In an alternative embodiment, the block polymer has the structure:

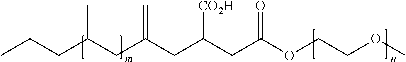

wherein m is 3 to 100, more preferably 10 to 30 and n is 3 to 100, more preferably 5 to 40.

The Unithox range of materials produced by Baker Petrolite have been found to be of use as block copolymers in the present invention. Examples of such block copolymers include Unithox® 720 (a block copolymer of polyethylene and polyethylene oxide having a molecular weight of 875 and an HLB value of 4), Unithox® 750 (a block copolymer of polyethylene and polyethylene oxide having a molecular weight of 1400 and an HLB value of 10) and/or X10044 (a block copolymer of polypropylene and polyethylene oxide having a molecular weight of 1300 and an HLB value of 4).

In an alternative embodiment of the invention, the amphiphilic copolymer is a cross-linked/network (or chain extended) copolymer. Copolymers of this type may be prepared using the same or similar polymer backbones to those described above in respect of amphiphilic graft copolymers.

In one embodiment of the invention, the amphiphilic copolymer is a cross-linked/network copolymer comprising a hydrophobic straight or branched chain carbon-carbon backbone having at least one hydrophilic side chain attached thereto.

### Method of Synthesis

Typically, the copolymers used in certain embodiments of the invention are synthesised by dissolving the backbone and graft in an organic solvent (e.g. toluene) and maintaining the mixture at reflux for a period of time sufficient to ensure reaction.

In another preferred embodiment, the synthesis is carried out in the absence of solvent, i.e. using a no-solvent approach using any mixing apparatus capable of mixing the (still viscous) molten MPEO/PEO side chain and backbone together. Preferably, the reaction temperature is from about 160 to about 180° C.

The reactions are preferably carried out under an inert gas to avoid oxidation of the polymers and hydrolysis of the maleic acid/anhydride groups.

In one preferred embodiment, the synthesis involves reacting from about 1 to about 4, more preferably, about 3 equivalents of side chain precursors with respect to each acylating group. Further details of the synthesis are described in WO 09/068,569 which is hereby incorporated by reference.

Preferably, the acylating group is derived from a maleic anhydride unit (either pendant to the backbone or within the backbone). Suitable side chain precursors which are polyether amines are available commercially; a range of mono and difunctionalised amine polymers of ethylene oxide (EO) and propylene oxide (PO) are sold under the Jeffamine brand name by Huntsman. Reaction between the amine functionalized polymers with maleic anhydride derived units, for instance, can generate any of the following structures:

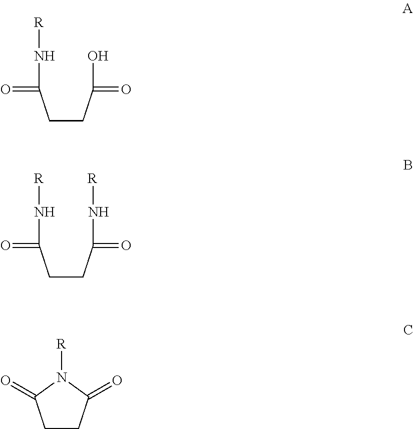

The structure denoted C may be formed by an intramolecular reaction of A, accompanied by the elimination of H₂O. This reaction is more likely to occur with the assistance of catalysis (e.g. by the addition of an acid). Both mono and difunctional amine polymers are suitable for use in the present invention. Depending on the reaction conditions, the use of hydrophilic difunctional amine side chain precursors can lead to a cross-linked or chain extended amphiphilic polymeric material. Alternatively, mono and difunctional side chain precursors may be combined to modify the properties of the resulting polymeric material as required. Jeffamine M-1000 and M-2070 are particularly preferred.

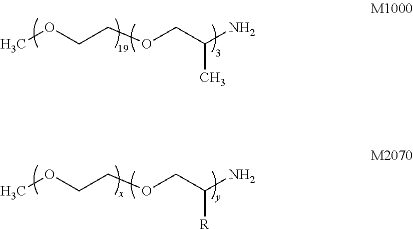

[wherein R═H for (EO), or CH₃ for (PO); x=6 (pure EO); y≈35 (EO and PO).]

Jeffamine M-1000 is a monoamine polyether with a EO:PO ratio of 19:3 and a molecular weight of approximately 1000. M-2070 is a monoamine polyether with an EO:PO ratio of 31:10 and a molecular weight of approximately 2000. Due to their relatively high levels of ethylene oxide they are regarded as hydrophilic materials. Both M-1000 and M-2070 have been found to react efficiently with PIP-g-MA.

In another preferred embodiment, the amphiphilic copolymer is prepared from the reaction of backbone precursors with a monoester of maleic anhydride, for example, to form PIP-g-MaMme (polyisoprene-graft-monoacid monomethyl ester supplied by Kuraray Co. Ltd, sold as LIR-410) with the general formula shown below:

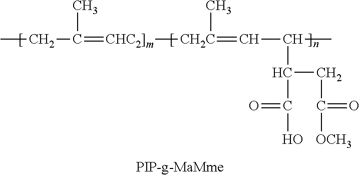

PIP-g-MaMme has a functionality (i.e. n) of approximately 10, an average molecular weight of about 25,000, and a glass transition temperature of −59° C. Each monomethyl ester may react with a single amine functionality.

As stated above, the properties of the amphiphilic copolymer depend not only on the character of the side chains grafted onto the carbon-carbon backbone, but also on the number of grafted side chains. In the present invention, one or more chain precursors react with each backbone precursor. More preferably, a plurality of side chain precursors react with each backbone precursor. The term “plurality” is defined herein as meaning more than one grafted side chain, i.e. more than one side chain precursor reacts with each backbone precursor.

In order to achieve the desired degree of hydrophilicity in the amphiphilic copolymer, it is preferred that the ratio of side chains to backbone repeat units in the resultant polymeric material is in the range of from about 1:500 to about 1:2, more preferably from about 1:350 to about 1:30. The side chains are preferably statistically distributed along the carbon-carbon backbone as the location of attachment of the side chain on the backbone will depend on the positions of suitable attachment locations in the backbone of the hydrocarbon polymer used in the manufacture.

When the side chains are linked to the polymer backbone via grafted maleic anhydride units, each maleic anhydride unit in the polymer backbone may be derivatised with either zero, one or two side chains.

In one preferred embodiment, the side chain precursors of general formula (I) or (II) comprise at least one nucleophilic group which is an amine. In the reaction to form an amphiphilic polymeric material, the nucleophilic groups react with pendant units on the polymer backbone which are acytating groups to form a polymeric material as defined above. Preferably, the pendant units are derived from maleic anhydride.

In one embodiment of the invention, each side chain precursor has two nucleophilic groups (for instance, X¹ is O or NR⁴) which may react with two acylating groups on different backbone precursor molecules, thereby forming a cross-linked structure. For example, a polyethylene oxide side chain is generally terminated with an alcohol at each end before derivatisation. Each alcohol may be grafted onto a maleic anhydride unit.

In some embodiments of the invention, where the acylating group is derived from maleic anhydride, only one side chain precursor reacts per maleic anhydride monomer. This leaves the unit derived from maleic anhydride with a free carboxylic acid group, which may be derivatised at a later stage in the method. This group may also be deprotonated to give an ionic pendant group in the polymeric material.

The reaction between the backbone precursors (for instance, PIP-g-MA) and the side chain precursors may be carried out in an organic solvent such as toluene. Typically, the reaction takes place at elevated temperatures, optionally in the presence of an activator for example, triethylamine. The yield may be increased by removal of the water from the reaction mixture by azeotropic distillation as toluene and water form azeotropic mixtures which boil at a lower temperature than any of the individual components.

The side chain precursor may also be reacted with a monoester derivative of PIP-g-MA, for example, the PIP-g-MaMme detailed above. The reaction of this monomethyl ester with the side chain precursor is typically carried out in an organic solvent such as toluene at elevated temperatures. Again, the yield of ester may be increased by removing water from the reaction mixture by azeotropic distillation.

Alternatively, the synthesis of the amphiphilic copolymer may achieved by mixing the intended side chain precursors with the backbone precursors in the absence of solvent. This ‘no-solvent’ process eliminates the costs associated with purchasing and handling organic solvents and removing otherwise harmful materials from the polymer. It will be appreciated that this approach is also desirable in eliminating volatile organic compounds that may be harmful to the environment. Further details of the no-solvent synthesis may be found in WO 09/050,203, the contents of which are hereby incorporated by reference.

The side chain and backbone precursors may be either in the form of a solid or in fluid form (e.g. in the form of a liquid or a gel), provided that they can be mixed easily. More preferably, the side chain and backbone precursors are either in the form of a liquid or finely ground solid. In one embodiment of the invention, the side chain precursors are in liquid form and the backbone precursors are in the form of a finely ground solid. More preferably, both the side chain and backbone precursors are in the form of a liquid at the temperature at which the acylation reaction takes place.

In one preferred embodiment of the invention, the backbone precursors are mixed with the side chain precursors by dissolving the backbone precursors in molten side chain precursors.

It will be appreciated by those skilled in the art that the reaction process may be performed using any apparatus that is capable of providing sufficient mixing. This includes reactors or other any vessels where agitation is provided, for example, by an overhead stirrer or a magnetic stirrer. More preferably, mixing is achieved using an appropriate extruder, z-blade mixer, batch mixer, U trough mixer, RT mixer, compounder, internal mixer, Banbury type mixer, two roll mill, Brabender type mixer, a wide blade mixer (or hydrofoil blade mixer), horizontal (delta or helical) blade mixer, kneader-reactor, or a variation thereof, such as a double z-blade mixer or twin screw extruder.

Increasing the temperature of the reaction mixture generally results in the side chain precursors melting, which allows more efficient mixing, and in turn contributes to an increase in the rate of reaction. The temperature of the reaction is preferably from about 50° C. to about 300° C., more preferably from about 100 to about 250° C., even more preferably from about 120° C. to about 200° C., and more preferably still, from about 140° C. to about 180° C. Preferably, the mixing apparatus is flushed with an inert gas to prevent degradation of the polymeric materials. Alternatively, the reactor may be placed under vacuum in order to ensure that air is excluded. The reaction can also be catalysed by the addition of acid or base. Optionally, water may be added to the reactor at the end of the reaction to hydrolyse any unreacted acylating groups. Advantageously, the hydrolysis of unreacted acylating groups can increase the hydrophilicity, and thus water compatibility or solubility, of the materials.

Any remaining acylating groups are preferably converted into acid groups by the addition of water to the material, or by an ageing process. An ageing process typically involves leaving the material in atmospheric air to ensure hydrolysis of any residual maleic anhydride by atmospheric moisture. Alternatively, the remaining acylating groups are hydrolysed with the aid of a base catalyst, or by the addition of an alcohol (hydroxyl) or amine with or without base. By way of an example, any remaining maleic anhydride groups are preferably converted into diacid groups by addition of water to the material.

The reaction mixture, at the end of the reaction, normally comprises unreacted starting materials which may include free side chain precursor and backbone precursor. There may also be some residual catalyst, if this has been used in the reaction. The reaction generally produces no by-products. The amphiphilic polymeric material need not be purified from the reaction mixture, since it can be advantageous to have free side chain precursors in the final composition. The free side chain precursor may interact with the amphiphilic polymeric material, thereby improving its properties.

Any PIP-g-MA of appropriate molecular weight distribution and maleic anhydride content will be suitable for the synthesis of the polymeric material. Alternatively, carboxylated PIP-g-MA materials in which the maleic anhydride is ring-opened to form a diacid or mono-acid/mono-methyl ester are also be suitable.

Preferably, the backbone precursors of the polymeric materials are derived from polyisoprene to which maleic anhydride has been grafted. By way of illustration, the level of grafting of MA is typically around 1.0 mol % in the PIP-g-MA. In PIP-g-MaMme, the level was 2.7 mol % of the mono-acid mono-methyl ester of MA. The level of grafting depends on the degree of functionalisation of the polyisoprene. For example, in PG1 (see below) the number of grafts per chain is generally between 1 and 7, whereas in PG2 (see below) it is between 1 and 10.

In one preferred embodiment, preferably from about 1 to about 4, more preferably from about 2 to about 3 equivalents of side chain precursors with respect to each maleic anhydride group are reacted. Reaction efficiency may be increased by reacting the PIP-g-MA used to synthesize PG1 with side chain precursors which are polyether amines. These are available commercially; a range of mono and difunctionalised amine polymers of ethylene oxide (E0) and propylene oxide (PO) are sold under the Jeffamine brand name by Huntsman.

When the backbone precursor of the amphiphilic polymeric material is a copolymer of maleic anhydride together with an ethylenically-unsaturated monomer, side chain precursors are typically terminated by an alcohol or amine nucleophilic group at one end and an alkyloxy group at the other. MeO-PEO-OH (MPEO) is an example of a preferred side chain precursor. In the method of formation of the polymeric material such side chains react with the maleic anhydride derived units via alcoholysis of the anhydride to give a carboxylic ester and carboxylic acid.

The reaction of maleic anhydride with an alcohol is an alcoholysis reaction which results in the formation of an ester and a carboxylic acid. The reaction is also known as esterification. The reaction is relatively fast and requires no catalyst, although acid or base catalysts may be used.

The net reaction may be represented as shown below. Pₓ and P_{y} represent the remainder of the copolymer/terpolymer and ROH is a representative side chain precursor.

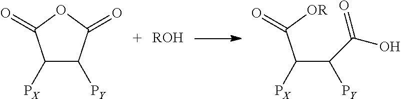

In one preferred embodiment, two side chains precursors represented by ROH may react at the same maleic anhydride monomer to give a compound of general formula

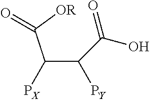

Alternatively, only one side chain precursor reacts per maleic anhydride monomer. This leaves the unit derived from maleic anhydride with a free carboxylic acid group, which may be derivatised at a later stage in the method. This group may also be deprotonated to give an ionic backbone in the polymeric material.

In one preferred embodiment, the side chain precursors may have hydroxyl or amine groups at each of their termini and each terminus reacts with a unit derived from maleic anhydride in different backbones to form a cross-linked polymeric material.

After reaction of the side chain precursors with a backbone precursor which comprises units derived from maleic anhydride in the backbone, any unreacted units derived from maleic anhydride in the backbone may be ring-opened. This may be performed by hydrolysis, or using a base. The resulting product may be ionisable.

This further reaction step has particular utility when there is a large proportion of maleic anhydride in the backbone, for instance in an alternating copolymer.

In one preferred aspect of the invention the backbone precursors comprise pendant units of general formula (IV),

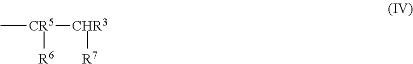

wherein R³ and R⁵ are each independently H or alkyl, and R⁶ and R⁷ are each independently H or an acyl group, provided that at least one of R⁶ and R⁷ is an acyl group, or R⁶ and R⁷ are linked to form, together with the carbon atoms to which they are attached, a group of formula (V),

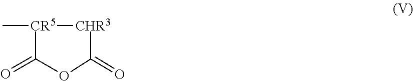

; with a side chain precursor of formula (VI)

HX¹—Y—X²P (VI)

wherein:

### X¹ is O, S or NR⁴;

### X² is O, S, (CH₂)ₚ or NR⁴;

p is 0 to 6;
each R⁴ is independently H or alkyl;
P is H or another backbone; and
Y is a hydrophilic polymeric group;
and in the method, the group HX¹ in the compound of formula (VI) reacts with the units of general formula (IV) or (V) to give the amphiphilic polymeric material wherein the side chains are of general formula (I)

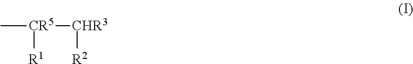

wherein R¹ and R² are each independently H, —C(O)WR⁴ or —C(O)Q;
provided that at least one of R¹ and R² is the group —C(O)Q;
or R¹ and R² together form a cyclic structure together with the carbon atoms to which they are attached, of formula (II)

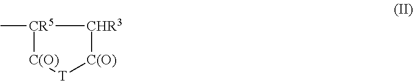

wherein:
R³ and R⁵ are each independently H or alkyl;

### W is O or NR⁴;

Q is a group of formula —X¹—Y—X²P;
T is a group of formula —N—Y—X²—P;

### X¹ is O, S or NR;

### X² is O, S, (CH₂)ₚ or NR⁴;

p is 0 to 6;
each R⁴ is independently H or alkyl;
P is H or another backbone; and
Y is a hydrophilic polymeric group.

In one preferred aspect of the invention the backbone precursors are part of the backbone itself (for example when maleic anhydride is part of the backbone), i.e.:

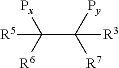

wherein each substituent has the definition set forth above.

The side chains thus have the general formula of R¹ and R² as defined above.

The side chains in the amphiphilic polymeric material thus comprise a unit derived from the acyl group of the backbone precursors.

The preferred substituents are the same as those given above for the preferred side chains in the polymeric material.

### Formulation of the Cosmetic Products

The cosmetic composition of the invention comprises a cosmetically acceptable diluent, excipient or carrier.

The amphiphilic copolymer of the present invention may be added to any known type of cosmetic composition. Preferably, the amphiphilic copolymers used in the present invention replace a portion or all of any wax, oil or a mixture thereof in a standard cosmetic formulation.

The cosmetic compositions of the present invention encompass lip care products (for example, a lipstick, lip gloss, lip liner, lip plumper, lip balm, lip sheer, lip ink, lip conditioner, lip primer or lip booster), face make-up products (for example, foundation, face powder, concealer, blusher and bronzer), and eye make-up products (for example, eye shadow, eyeliner or mascara).

It is important when producing cosmetic compositions they should be produced using materials that are safe and currently comply with the regulatory and legal requirements in the markets. Cosmetic materials are classified using the International Nomenclature of Cosmetic Ingredients (INCI) system. The INCI system (as of November 2009, administered by the Personal Care Products Council) is designed to make easy identification of ingredients used in personal care and cosmetic products. It will be understood by those skilled in the art that generally speaking it will be possible to replace a material with one from a different manufacturer with an identical INCI name without notably changing the resulting product.

Preferably, the cosmetic composition of the invention further comprises one or more of an emollient, a colorant, a moisturizer, a UV blocker, an active agent, an antioxidant, a vitamin, a lip plumping agent (for lip products only), a fragrance, a flavour or flavourant, a sweetening agent, a vegetable or herb extract and/or a preservative.

In one preferred embodiment, a colourant is incorporated to the formulations of the invention. Preferably, the colourant is in the form of an inorganic pigment (e.g. a metal oxide) or dye that is added to the formulations to impart colour to the product and the lips.

For lip products, additional agents may be added to the formulations to increase the gloss of lips, for example, by increasing their reflectivity). These may typically find use in lip gloss lipstick or even lip bairn products designed to deliver gloss to the lips and may include by way of example mother of pearl and mica or calcium sodium borosilicate glass coated with a metal oxide such as iron oxide or titanium dioxide in an appropriate particulate form, or alternatively oils and waxes or combinations thereof that accomplish this effect. Lip formulations may optionally include any agent that has a temporary or permanent lip plumping effect, for instance menthol, chilli, vanillyl butyl ether or a peptide based material such as for instance hexapeptide-3Temporary lip plumping agents may optionally work by causing irritation to the lip tissue, whereas longer lasting or more permanent effects may be observed from agents that modify the collagen or moisture composition of lips, for example. Optionally, the amphiphilic graft copolymer may be mixed with the lip plumpling agent before addition to the cosmetic composition to modify the agents behaviour on the lips, for instance extending the time a temporary lip plumping agent will give a detectable benefit. Formulations may also contain an opacifying or pearlescent material.

A number of other components may be incorporated into cosmetic compositions to increase the range of benefits that the product is able to offer. These include emollients and moisturisers such as aloe vera, cocoa butter, squalane, Coenzyme Q-10, allantoin, sunscreens and other agents capable of blocking or assisting to block the harmful effect of the sun's light (including organic materials like oxybenzone, Padimate O or octinoxate or alternatively inorganic materials like zinc or titanium dioxide, or a combination of both inorganic and organic materials), and active ingredients capable of, or perceived, to benefit the health or appearance of the skin or to treat a disease. These ingredients include, but are not limited to, antioxidants and/or vitamins (e.g. vitamin E and its derivatives), hyaluronic acid, analgesics (e.g camphor, menthol, phenol), collagen and its derivatives. Antioxidants such as vitamin E or butylhydroxytoluene (BHT) may be also be added to avoid the composition being spoiled or altered by the oxidative degradation of its components.

Optionally, but preferably formulations may also include fragrances and/or flavours (such as fruit, herb, vegetable, savoury or confectionary flavours) and/or sweeteners (for instance saccharin), to enhance the sensorial profile of the product making its application or use more pleasant and appealing to the consumer or user. For instance, the flavour or fragrance may be used to counteract and mask the intrinsic flavour or fragrance of the oils and waxes used in the composition or might be used to give the user the perception of a flavour on the lips. Vegetable, and particularly herb, extracts are frequently added. Formulations may optionally include egg white. Formulations may also include the use of a preservative to prevent the growth of bacteria and/or fungi in the compositions, for instance the family of alkyl parabens including methyl, ethyl, propyl and butyl paraben, diazolidinyl urea, sodium or potassium benzoate. It will be understood by those skilled in the art that some ingredients that may be added to preserve or assist in the preservation of the composition have another primary or secondary role, e.g. as an emollient. Formulations may include an agent to increase the stability of the lip product structure or the compatibility of the agents therein. Formulations may also contain film forming agents for instance clays or modified clays and polymers including stearalkonium hectorite and polybutene.

In the case of alcohol and water based formulations, for example, certain foundation and lip ink formulations, the polymer is typically dissolved or dispersed in one of the phases and then mixed with the other components of the formulation. In many cases it is easier to achieve this process by raising the temperature of the mixture, for instance to 80° C. in the case of water. It may also be advantageous to add an emulsifier to ensure better compatibility with the mixture. In the case of more hydrophobic copolymers it is often advantageous to dissolve or disperse them in the ethanol phase first if present. Water based foundation formulations may also include clays, the polymer may optionally be premixed with the clays first to increase their performance. An additional polymer system may be added to water based formulations (for instance lip make-up or foundation) to increase further their transfer resistance. Formulations may optionally include various natural or synthetic viscosity modifiers or thickeners for instance polymers like poly(acrylic acid) (marketed as carbomer), cellulosic materials like hydroxyethylcellulose, natural gums like xanthan and guar gum or clays or clay derivatives such as stearalkonium hectorite.

A useful guide to formulations that may typically be adapted to use the amphiphilic copolymer may be found in “A Formulary of Cosmetic Preparations Volume. One: Decorative Cosmetics, ed. Anthony L. L. Hunting, 2^{nd} edition 2003, Micelle Press, Weymouth, England, the contents of which is incorporated as an example.

In one preferred embodiment of the invention, the cosmetic composition is in the form of an emulsion, preferably, a water-in-oil or oil-in-water emulsion.

A further aspect of the invention relates to the use of an amphiphilic polymer as described above in the preparation of a cosmetic composition.

### Formulation of the Lip Products

Preferably, for lip products, the cosmetically acceptable diluent, excipient or carrier is selected from an oil, a fat and a wax, or most preferably a mixture thereof.

Most lip products (with the exception of lip stain) use mixtures based on oils, fats and/or waxes sometimes referred to as structuring agents or ingredients.

The cosmetic compositions of the present invention are typically prepared by melting the above-described polymer with one or more oils and/or waxes, resulting in a homogenous product with all the desirable properties expected of the product.

A further aspect of the invention therefore relates to a process for preparing a cosmetic composition, said process comprising melting an amphiphilic copolymer as described above with one or more oils and/or waxes to form a homogenous product.

A further aspect of the invention relates to a cosmetic composition obtainable by the above described process.

In the context of the present invention, an oil is encountered at room temperature (between about 21° C. about 25° C.) as a non-aqueous liquid and a fat a paste like or fluid to semi-fluid consistency, whereas a wax is a material that has some plasticity at normal ambient temperatures and a melting point above about 45° C. to 50° C. The materials used in the formulations of the present invention are of a sufficiently non-toxic nature that they can be used in the required concentrations in the final product. The materials also need to be cosmetically acceptable, although some problems (e.g. poor taste or smell or undesirable colour) can be overcome by judicious formulation by those skilled in the art.

Suitable waxes include, but are not limited to, those derived from vegetable, animal, petroleum, synthetic or mineral sources. Preferred examples include ozokerite, microcrystalline wax, beeswax, carnauba, lanolin and candelila waxes.

Suitable oils, fats and waxes include, but are not limited to, those derived from vegetable, animal, petroleum, synthetic or mineral sources. Preferred examples include castor, olive, jojoba, coconut, sesame, safflower, orange, mineral, canola and various silicone/methicone oils, lanolin and petrolatum. Many of theie oils will remain on the lips during normal use. Some fatty ingredients like shea butter or lanolin may possess emollient properties as well. Optionally volatile oils (frequently based on dimethicone. for instance) may be used instead of, or in conjunction with, other oils. These oils evaporate on the surface of the lips, increasing the transfer resistance of the lip product, most typically lipstick.

Some of these materials are multifunctional and also impart other benefits. For example, lanolin, petrolatum, dimethicone are recognized for their benefits in skin protection. In general the materials are adjusted to build the required physical properties of the lip product. This is typically achieved by varying the proportion of hard (higher melting/drip point) and soft (lower melting point) waxes and oils. For example, in a product like lipstick, a harder physical form is needed and a higher ratio of wax to oil is used. In a more fluid product like lip gloss, a higher proportion of oils or soft waxes is used.

Lip liner may be dispensed from a solid pencil, made from a material like wood as an outer surface for example. The amphiphilic copolymer and other ingredients may optionally be formed by mixing the materials in together in a granulated form and compressing them together. Preferentially the materials will be mixed by melting them together and intimately mixing them together. The components may also be combined by a method in which the components are continuously passed into an extruder and fed out of the other side.

The physical properties of the preferred amphiphilic graft copolymers (e.g. PG1) may be regarded as most similar to those of a wax as they have a melting point centred above that typically encountered at ambient temperature. Lip products like lipstick are typically manufactured by combining the relevant oils, fats and waxes together at elevated temperature and subsequently mixing with a suitable stirrer when the waxes are sufficiently soft. The graft copolymer may be added to the mixture in a similar manner. To aid the dispersion of the material, it is typically used in either pellet or granulated or powdered form. In some cases it can be advantageous to elevate the temperature prior to addition to the mixture to make homogenisation easier. The copolymer can also be combined with a suitable oil (e.g. castor oil) or wax (e.g. bees wax) to make it easier to manipulate. For example, the product with an oil may be a paste, whereas with a wax, the product may be a faster melting solid.

The temperature and mixer are chosen such that an essentially homogenous mixture of the ingredients can be efficiently formed without wasting energy or substantially degrading the components. Optionally, a specially designed lipstick kettle may be used. Preferably, the temperature is in the region of from about 50° C. to about 120° C., more preferably, about 70 to about 90° C. This temperature may be maintained whilst the other ingredients (pigment, actives etc) are added and mixed in. If necessary, the temperature may be reduced to avoid excess evaporation or degradation of the fragrances or active ingredients added. Alternatively, it is possible to premix fragrances or active ingredients with the structural components before heating the mixture. For example, it is possible to premix the amphiphilic copolymer with a fragrance, flavour or active ingredient to ensure good distribution of the agent within the final product as well as longer lasting retention of the component on the lips.

In many cases, for example, with lip balm, the final mixture is preferably poured in molten form into the final package that is used to market the product to consumers. In the case of lipstick, the mixture is preferably poured into a specially designed mould. These are typically constructed from a thermally robust material such as metal and many designs are available commercially with varying capacities. The mould is typically designed to impart a cylindrical shape to the final lipstick. The mould can be designed to shape the tip of the lipstick as desired, with different designs being familiar to those skilled in the art. The lipstick mould is frequently heated to match that of the mixture being poured in to avoid too rapid solidification and defects in the resulting stick. After addition of the hot mixture, it is then allowed to cool, resulting in solidification of the mixture. Optionally, the mould and mixture may be cooled to sub ambient temperatures, thereby increasing the speed at which it sets. The lipstick is then removed from the mould and placed inside the tube in which it is to be marketed.

Preferably, from about 1 to about 20%, more preferably, from about 2.5 to about 15%, more preferably from about 2.5 to about 12% by weight of the polymer is used in a hard lip product like lipstick. In a soft lip product like lip balm, preferably from about 1 to about 50%, more preferably from about 5 to about 40%, more preferably still from about 5 to about 35% by weight of the polymer is added. Due to the lower hardness of soft lip balm products it can be advantageous to take advantage of the ability to incorporate more polymer into the formulation. In some cases however it will be preferable to use a lower amount of polymer to allow the incorporation of more oil.

Preferably, the cosmetic composition of the invention is a lip product, more preferably, a lipstick, lip gloss, lip liner, lip plumper, lip balm, lip sheer, lip ink, lip conditioner, lip primer or lip booster.

In a preferred embodiment of the invention there is provided a lipstick, lip gloss or lip balm base comprising:
(i) An amphiphilic graft copolymer, such as PG1, PG2, PG3, PG4, PG5, PG6 or PG7 as defined herein, preferably PG1, in an amount of from about 1% to about 40% by weight;
(ii) One or more waxes, oils and fats in an amount from about 10% to about 50% by weight. Most preferably these are typically from natural, mineral or synthetic origin such as ozokerite wax, microcrystalline wax, beeswax, carnauba wax, or candelila wax; lanolin, shea butter, castor oil, olive oil, jojoba oil, coconut oil, sesame oil, safflower oil, orange oil, mineral oil, canola oil, a silicone/methicone oil, or paraffin oil; and optionally
(iii) One or more ingredients with some form of antioxidant, and/or biocidal/preservative action, for instance vitamin E, vitamin E acetate, Vitamin C, BHT, an alkyl paraben such as propylparaben, methyl paraben or a mixture of parabens, diazolidinyl urea, sodium and/or potassium benzoate in an amount from about 0.1% to about 1% by weight.

In a preferred embodiment of the invention there is provided a lipstick, lip glossor lip balm base comprising:
(i) An amphiphilic block copolymer, such as PB1, PB2 or PB3 as defined herein, preferably PB1, in an amount from about 1% to about 40% by weight;
(ii) One or more waxes, oils and fats in an amount from about 10% to about 50% by weight. Most preferably these are typically from natural, mineral or synthetic origin such as ozokerite wax, microcrystalline wax, beeswax, carnauba wax, or candelila wax; lanolin, shea butter, castor oil, olive oil, jojoba oil, coconut oil, sesame oil, safflower oil, orange oil, mineral oil, canola oil, a silicone/methicone oil, or paraffin oil; and optionally
(iii) One or more ingredients with some form of antioxidant, and/or biocidaUpreservative action, for instance vitamin E, vitamin E acetate, Vitamin C, BHT, an alkyl paraben such as propylparaben, methyl paraben or a mixture of parabens, diazolidinyl urea, sodium and/or potassium benzoate in an amount from about 0.1% to about 1% by weight.

Optionally, the lipstick, lip gloss lip balm further comprises one or more additional suitable excipients, such as pigments, flavours, fragrances, sweeteners, UV actives and/or gloss enhancing agents.

### Formulation of the Face Products

Face cosmetics containing the amphiphilic copolymers can be formulated into a variety of formats including liquids, powders, creams, sticks or gels according to that desired. Many of the previously discussed cosmetic ingredients used in lip products including oils waxes, pigments and fragrances may be used.

Foundation formulations may contain a range of different components depending on the physical format required of the final product. In the case of a fluid any of the oils or emolients discussed previously. The polymer may thus be mixed in at elevated temperature in a similar manner to that described previously for lip products.

In the case of a foundation that is an emulsion of water and oil, the polymer will typically be mixed into the oil phase, preferably with a little wax prior to addition to the mixture. Alternatively, it may be preferably added to the formulation when the oils and waxes are added. In the case of a more viscous product like a cream, viscosity modifiers may be added to increase the viscosity of the resulting formulations.

In the case of a powder formulation, the foundation will be typically based on talc, optionally mixed with a range of powdered ingredients like kaolin, precipitated chalk, titanium dioxide, zinc oxide, zinc stearate, bismuth oxychloride, magnesium carbonate and magnesium stearate. In addition to acting as fillers, many of these powdered materials have secondary benefits such as preventing caking in the product (magnesium and zinc stearate) or opacifiers (magnesium carbonate). It is preferred that the polymer be premixed with the talc or powdered materials, either by adding as a melt and grinding the resulting material to the desired size or by combining the materials in a volatile medium and coating them with a suitable process such as spray drying.

It is a preferred embodiment that the polymer is mixed in with an oil or wax if present prior to being added to the foundation.

If it is desired that the resulting product be marketed as a mineral foundation, an inorganic material like zinc oxide, titanium dioxide or bismuth oxychloride will typically be added.

In the case of a concealer, a liquid format is typically preferred. A mixture of the oils and waxes mentioned before will typically be used. The amphiphilic copolymer may thus be typically mixed into the waxes and oils as with foundation. The formulation will typically be formulated with a greater level of pigment than is typical for other cosmetic products to enable it to give coverage over blemishes. This is most commonly preferably using comparatively large amounts of titanium dioxide, typically 10 to 30 by weight percent of the formulation.

Rouge or blusher may come in a range of formats including powder, cream or fluid. The amphiphilic copolymer may typically be mixed in a similar method to that in foundation. Frequently a red pigment (e.g. iron oxide, preferably 0 to 15%) will be incorporated to give a hint of red colour to the cheeks and face.

In addition to these materials and the amphiphilic polymer the formulation may optionally include any of the other types of cosmetically acceptable materials listed in this patent, including but not limited to waxes, oils, emulsifiers, a further agent to control adhesion, agents to give skin protection from ultraviolet radiation, antioxidants, preservatives, fragrances, and pigments.

Preferably, from about 1 to about 20%, more preferably, from about 1 to about 15%, more preferably from about 2 to about 12% by weight of the polymer is used in face products.

### Formulation of the Eye Products

Eye cosmetics containing the amphiphilic copolymers are typically formulated into liquids, powders, or gel formats according to that desired. Many of the previously discussed cosmetic ingredients used in lip products including oils waxes, pigments and fragrances may be used. One exception is that the purity and safety of all components, particularly pigments, is of great importance.

Mascara may generally be divided into water based and non-water based transfer resistant formulations. The water based formulations will typically include surfactants to help solubilise the hydrophobic components of the system. Transfer resistant formulations will contain a volatile oil that will evaporate comparatively quickly to result in a transfer resistant film. Optionally, a mascara formulation may contain a fibre like nylon or rayon to increase the length of the lashes, it is a preferred aspect of the invention that the amphiphilic polymer may be premixed with the fibre to increase its adhesion. Preferably the amphiphilic polymer will be formulated into a mixture of compatible oils and waxes at elevated temperature and then added to the formulation with good stirring.

Eye shadow is preferentially formulated by dispersing intensely coloured inorganic pigments into a liquid or cream base in a similar manner to that used with liquid foundation. In another preferred method the amphiphilic copolymer is mixed in with solid ingredients like talc to make a powder formulation, using the methods discussed for making powder foundation.

Eyeliner may be dispensed from a solid pencil, made from a material like wood as an outer surface for example. The amphiphilic copolymer and other ingredients may optionally be formed by mixing the materials in together in a granulated form and compressing them together. Preferentially the materials will be mixed by melting them together and intimately mixing them together. The components may also be combined by a method in which the components are continuously passed into an extruder and fed out of the other side.

In addition to these materials and the amphiphilic polymer the formulation may optionally include any of the other types of cosmetically acceptable materials listed in this patent, including but not limited to waxes, oils, emulsifiers, a further agent to control adhesion, agents to give skin protection from ultraviolet radiation, antioxidants, preservatives, fragrances, and pigments.

Preferably, from about 1 to about 20%, more preferably, from about 1 to about 15%, more preferably from about 2 to about 12% by weight of the polymer is used in eye products.

### Preferred Copolymers of the Invention

The following copolymers were prepared according to the methodology set forth below.

| | | Amount of | | Amount of | Amount of |
|---|---|---|---|---|---|
| | | MPEO 2K | Amount of PEO | Jeffamine M-2070 | MPEO 350 |
| Polymer | Backbone | (equivalents)¹ | 2K (equivalents)¹ | (equivalents)¹ | (equivalents)¹ |
| PG1 | PIP-g-MA | 2.8 | 0 | 0 | 0 |
| PG2 | PIP-g-MA | 1 | 0 | 0 | 0 |
| PG3 | PIP-g-MA | 1 | 1.8 | 0 | 0 |
| PG4 | PIP-g- | 1 | 0 | 0 | 0 |
| | MaMme | | | | |
| PG5 | PIP-g-MA | 0 | 0 | 2 | 0 |
| PG6 | PIB-alt- | 0.3 | 0 | 0 | 0 |
| | MA | | | | |
| PG7 | C18-alt- | 0 | 0 | 0 | 0.5 |
| | Ma | | | | |

PIP = polyisoprene;
g = graft;
MA = maleic anhydride;
MaMme = monoacid monomethyl ester;
PIB-alt-MA = Poly(isobutylene-alt-maleic anhydride);
C18-alt-MA = Poly(maleic anhydride-alt-1-octadecene):
MPEO = methoxy poly(ethylene oxide);
PEO = poly(ethylene oxide);
K = 1000 molecular weight units.
¹By number of equivalents of graft (e.g MPEO, PEO or Jeffamine) in the table above, it should be understood that the ratio of graft to each unit of maleic anhydride in the backbone is indicated. For instance in the case of PG1, 2.8 equivalents of MPEO 2K were added relative to each unit of maleic anhydride. An excess of MPEO will therefore be present in the final product. In the case of PG6, 0.3 equivalents of MPEO 2K were added to each maleic anhydride unit and thus a maximum of 30% of the maleic anhydride will have reacted with graft. The PIP-g-MA has an average of 2-5 maleic anhydride units per chain, or typically less than 2% by weight, are available for reaction with grafts. The PIB-alt-MA backbone is an alternating copolymer of isobutylene and maleic anhydride, and thus by contrast has between 60 and 70 weight percent of maleic anhydride. Thus, more MPEO is actually added to PG6 than PG1 and the resulting polymer would be expected to have a higher HLB than PG1, in other word be more hydrophilic.

In a preferred embodiment of the invention the amphiphilic copolymer is PG1, PG2, PG3, PG4, PG5, PG6 or PG7, or a mixture thereof.

Block copolymers of ethylene and ethylene oxide (Unithox 720, Polymer PB1) and

(Unithox 750, Polymer PB2), or propylene and ethylene oxide (X¹⁰⁰⁴⁴, Polymer PB3) manufactured by Baker Hughes were placed in the cosmetic compositions. PB1 has a melting point (m.p.) of 106° C., an HLB (hydrophobic lipophilic balance) of 4 and a molecular weight of 875. Unithox has a m.p. of 107° C., an HLB of 10 and a molecular weight of 1400. X10044 has a m.p. of 107° C., an HLB of 4 and a molecular weight of 1300.

| | Hydrophobic | Hydrophilic | Molecular | | |
|---|---|---|---|---|---|
| Polymer | Block | Block | Weight | HLB | Trade Name |
| PB1 | Polyethylene | PEO | 875 | 4 | Unithox 720 |
| PB2 | Polyethylene | PEO | 1400 | 10 | Unithox 750 |
| PB3 | Polypropylene | PEO | 1300 | 4 | X10044 |

In a preferred embodiment of the invention the amphiphilic copolymer is PB1, PB2 or PB3, or a mixture thereof.

A preferred method of measuring the molecular weights of the polymeric materials used in the present invention is set forth below. Alternative methods are well known in the art and may be found in J.M.G. Cowie, Polymers: Chemistry and Physics of Modern Materials

### Reference Method A: Determination of Molecular Weights of Polymeric Materials and Free MPEO

The polymer samples were analyzed using a PL-GPC50plus GPC system manufactured by Polymer Labs. The following conditions were used:

Eluent: THF stabilised with 250 ppm BHT

### Eluent RI: 1.408

Flow Rate (ml/min):1

### Temperature: 40° C.

### Column Set Name: 2 Columns 30 mm PL gel 5 um MIXED-D

### Detector Name: DRI

### Detector Calibration Curve: Polystyrene Standards (538Da-265000Da)

This apparatus was used to determine the molecular weights of all of the copolymers.

In order to determine the amount of any free MPEO present in a samples was 10 different solutions of known concentration of MPEO 2000 in THF (0.05-2 mg/mL) were accurately prepared and analysed on the apparatus. The relevant intensity of the samples was then used to generate a calibration curve which was used to generate the concentration of free MPEO in the samples.

Reference Method B: Determination of Degrees of Grafting with PEG using FT-IR

The analysis was carried out on a PerkinElmer Paragon 2000 Infrared spectrometer. Samples for analysis were dissolved in spectrometric grade chloroform and placed in a liquid cell (Barium fluoride plates separated by PTFE spacer) in a mounting bracket/carriage in an IR beam with known cell path length.

A sample of the batch of the backbone used to synthesize the graft copolymer was accurately weighed out ˜0.1 g (+/−0.05 g) into the stoppered conical flask and dissolved in 10 g of accurately weighed out chloroform. The FT-IR of the sample was collected, and the percentage transmission values measured at 1830 cm⁻¹ and at 1790 cm⁻¹ recorded. The sample of the final graft copolymer was accurately weighed out ˜1.5 g (+/−0.5 g) into the stoppered conical flask, dissolved in 10 g of accurately weighed out chloroform, and studied by FT-IR in a similar manner. The concentration of maleic anhydride in each sample was then calculated using the following formula:

$$μ mole / g ( in sample ) = \frac{33600}{C} \times {Log}_{10} \frac{\% T ( at 1830.0 {cm}^{- 1} )}{\% T ( at 1790.0 {cm}^{- 1} )}$$

where C is the concentration in the test solution (quoted in mg g⁻¹). The percentage conversion of maleic anhydride can then be determined by comparing the values from the backbone and graft copolymer.

### Reference Method C: Determination of the Strength of Lipstick

As mentioned earlier careful choice of the quantities of oil and wax in the product are necessary to ensure the physical state of the final product matches that required of the final product. For the more solid products (e.g. lipstick and hard lip balms) the strength of the final product is also measured to ensure it will not fracture during normal use. Various apparatus for testing the strength of formulated lip products is known to those skilled in the art, and many such devices may be adapted to measure the strength of the sticks (for instance tensiometers). The strength of the lipstick samples or lip balm in stick format described in the following examples was measured using a simple method as described below.

The sample to be tested must be a final product in a lipstick tube manufactured using the methods described later. As temperature affects the hardness of the sticks, the samples were placed in an oven maintained at 25° C. for a minimum period of 1 h to equilibrate before the test. The lipstick under test was fully extended from the tube and the tube itself clamped using a metal retort stand. A plastic bucket of known weight was placed on top of the fully extended lipstick and the bucket filled slowly with water. The test was complete at the point at which the lipstick breaks. The total mass of bucket is determined from the weight of the bucket and the amount of water added. The test was repeated a total of three times and the average weight required to break the stick recorded as a measure of its hardness.

It was generally found that the addition of the amphiphilic copolymer to the mixture did not adversely affect the strength of the resulting sticks. In case the properties of the lipstick are found to not be optimal the strength can still be adjusted by varying the proportion of wax or oil in the formulation, for instance by adding a greater quantity of harder wax if the formulation is too soft, or oil if it is too hard.

The present invention is further described with reference to the following non-limiting Examples.

### EXAMPLES

Any parameters calculated or studies carried out in the examples are done using the appropriate reference method set forth above.

### Example 1

### Reaction of Polyisoprene-Graft-Maleic Anhydride with methoxy poly(ethylene oxide) (Preparation of PG1) in a Reaction Flask

PIP-g-MA (300 g, Polyisoprene-graft-maleic anhydride obtained from Kuraray, LIR-403 grade) having the CAS No. 139948-75-7, an average M_{w} of approximately 25,000 and a typical level of grafting of MA of around 1.0 mol %, and methoxy poly(ethylene oxide) (MPEO) (212 g, purchased from Clariant), having an average molecular weight of 2000 were weighed out and added to a reaction flask with a 1 L capacity, equipped with an overhead stirrer. A flow of nitrogen gas was passed through the vessel, which was then heated to 120° C. using an oil bath. Stirring of the molten mixture then commenced and the vessel was heated to 160° C.

The reaction mixture was maintained at this temperature for a total of approximately 24 hours. Following this it was allowed to cool to below 100° C. and water (400 mL) was then added. The mixture was allowed to cool to room temperature and the water was removed by filtration, after which the product was dried under vacuum at 40-50° C.

The product was studied using GPC and FTIR.

### Example 2

### Reaction of Polyisoprene-Graff-Maleic Anhydride with methoxy poly(ethylene oxide) (Preparation of PG1) in a High Shear Granulator mixer

PIP-g-MA (738 g, Polyisoprene-graft-maleic anhydride obtained from Kuraray, LIR-403 grade) having the CAS No. 139948-75-7, an average M_{w} of approximately 25,000 and a typical level of grafting of MA of around 1.0 mol %, and methoxy poly(ethylene glycol) (MPEO) (526 g, purchased from Clariant), having an average molecular weight of 2000 were weighed out and added to a Lodige 3 L batch mixer, equipped with an overhead stirrer. A flow of nitrogen gas was passed through the vessel, which was then heated to 120° C. using an oil bath. Stirring of the molten mixture then commenced and the vessel was heated to 160° C.

The reaction mixture was maintained at this temperature for a total of approximately 24 hours. Following this it was allowed to cool to below 100° C. and water (1 L) was then added. The mixture was allowed to cool to room temperature and the water was removed by filtration, after which the product was dried under vacuum at 40-50° C.

The product was studied using GPC and FTIR.

### Example 3

### Reaction of Polyisoprene-Graft-Maleic Anhydride with methoxy poly(ethylene glycol) (Preparation of PG1) in a z-Blade Mixer

PIP-g-MA (738 g, Polyisoprene-graft-maleic anhydride obtained from Kuraray, LIR-403 grade) having the CAS No. 139948-75-7, an average M_{w} of approximately 25,000 and a typical level of grafting of MA of around 1.0 mol %, and methoxy polyethylene oxide) (MPEO) (526 g, purchased from Clariant), having an average molecular weight of 2000 were weighed out and added to a Lodige 3 L batch mixer, equipped with an overhead stirrer. A flow of nitrogen gas was passed through the vessel, which was then heated to 120° C. using an oil bath. Stirring of the molten mixture then commenced and the vessel was heated to 160° C.

The reaction mixture was maintained at this temperature for a total of approximately 24 hours. Following this it was allowed to cool to below 100° C. and water (0.5 L) was then added. The mixture was allowed to cool to room temperature and the water was removed by filtration, after which the product was dried under vacuum at 40-50° C.

The product was studied using GPC and FTIR.

### Example 4

### Reaction of Polyisoprene-Graft-Maleic Anhydride with methoxy poly(ethylene oxide) (Preparation of PG2) in a z-Blade Mixer

Polymer PG2 was prepared in a similar manner to Example 3 using PIP-g-MA (4.0 Kg,) and MPEO (1.23 Kg). The reaction was maintained at a temperature of 150° C. for 2 hours, the reaction mixture was then allowed to cool to 90° C. and mixing maintained for further 5 hours. After a total of 7 hours the product was discharged at about 80° C. and allowed to cool to ambient temperature.

The structure was confirmed using GPC and FTIR.

### Example 5

### Reaction of polyisoprene-Graft-Maleic Anhydride with Methoxy poly(ethylene oxide) (Preparation of PG3) and Combination with Poly(ethylene oxide) in a Z-Blade Mixer

Polymer PG3 was prepared in a similar manner to that in Example 4 using PIP-g-MA (3.2 Kg, Polyisoprene-graft-maleic anhydride) and MPEO (1.03 Kg). However, after reaction of the MPEO was complete, poly(ethylene oxide) (PEO, 1.86 Kg) was introduced and the mixture allowed to stir for a further 4 hours at 90° C., before being allowed to cool to ambient temperature.

The structure was confirmed using GPC and FTIR.

### Example 6

### Reaction of Polyisoprene-Graft-Monoacid Monomethylester with poly(ethylene glycol) methyl ether (Preparation of PG4) in a z-Blade Mixer

Polymer PG4 was prepared in a similar manner to that used in Example 4. PIP-g-MaMe (300 g, Polyisoprene-graft-maleic acid methyl ester obtained from Kuraray, LIR-410 grade) having the CAS No. 139948-75-7, an average M_{w} of approximately 25,000 and a typical level of grafting of maleic anhydride of around 10 mol %, and MPEO (240 g, purchased from Clariant), having an average molecular weight of 2000 were weighed out and added to a Z blade mixer. A flow of nitrogen gas was passed through the vessel, which was then heated to 120° C. using a hot oil jacket. Mixing of the molten mixture then commenced and the vessel was then heated to 160° C. for 5 hours, the reaction mixture was then allowed to cool to 90° C. and mixing maintained for further 2 hours. After a total of 7 hours the product was discharged from the z-blade mixer at about 80° C. and allowed to equilibrate to ambient temperature.

### Example 7

### Reaction of Polyisoprene-Graft-Maleic anhydride with an Amine Functionalised Polyether (Jeffamine M-2070) (Preparation of PG5) with a 2:1 Ratio of Graft to Each Maleic Anhydride Group

This product was prepared using the same methodology as Example 1 using LIR-403 (500 g) and an amine functionalised polyether (Jeffamine M-2070, 290.0 g), and a 1 L reaction flask. The structure was confirmed using GPC and FTIR.

PIP-g-MA (500 g, Polyisoprene-graft-maleic anhydride obtained from Kuraray, LIR-403 grade) having the CAS No. 139948-75-7, an average M_{w} of approximately 25,000 and a typical level of grafting of MA of around 1.0 mol %, and an amine functionalised polyether (Jeffamine M-2070, 290.0 g, obtained from Huntsman), having an average molecular weight of 1000 were added to a reaction flask with a 1 L capacity, equipped with an overhead stirrer. A flow of nitrogen gas was passed through the vessel, which was then heated to 120° C. using an oil bath. Stirring of the molten mixture then commenced and the vessel was then heated to 160° C.

The reaction mixture was maintained at this temperature for a total of approximately 24 hours. Following this it was allowed to cool to room temperature.

The structure was confirmed using GPC and FTIR.

### Example 8

### Reaction of Poly(isobutylene-alt-maleic anhydride) with methoxy poly(ethylene oxide) of Mₙ: 2000 g mol⁻¹ (Preparation of PG6)

Solvents were obtained from Fisher Scientific and were of HPLC (high performance liquid chromatography) or similar quality.

Polyisobutylene-alt-maleic anhydride) (Mₙ: 6000 g mol⁻¹, 60 g, supplied by Sigma-Aldrich) and methoxy poly(ethylene oxide) (Mₙ: 2000 g mol⁻¹, 220 g manufactured by Clariant) were dissolved in a mixture of dimethylformamide (DMF, 200 mL) and toluene (200 mL) in a reaction flask. The flask was heated at reflux temperature under nitrogen gas for 48 h, any water present being removed from the reaction by means of azeotropic distillation and collection into a Dean-Stark apparatus. The resulting polymer solution was then allowed to cool to room temperature, diluted with more toluene (200 mL) and precipitated into diethyl ether (500 mL). The polymer was then recovered using filtration, washed with more diethyl ether and dried to remove traces of solvent to yield solid PG6.

The grafting of MPEO onto the backbone was confirmed using infra-red spectroscopy.

### Example 9

### Reaction of poly(maleic anhydride-alt-1-octadecene) with methoxy poly(ethylene oxide) of Mₙ: 350 g mol⁻¹ (Preparation of PG7)

Poly(maleic anhydride-alt-1-octadecene) (PA18-LV grade, 20-25 000 g mol⁻¹, 116.7 g, manufactured by Chevron-Philips International) and methxoy poly(ethylene oxide) (Mₐ: 350 g mor, 58.3 g supplied by Sigma-Aldrich) were dissolved in toluene (400 mL, HPLC grade, Fisher Scientific) in a reaction flask. The flask was heated at reflux temperature under nitrogen gas for 60 h, any water present being removed from the reaction by means of azeotropic distillation and collection into a Dean-Stark apparatus. The resulting polymer solution was then allowed to cool to the point it could be safely added to a round bottom flask. The solvent was then removed under vacuum using a rotary evaporator. The polymer was then dried in a vacuum oven to remove traces of solvent yielding PG7.

The grafting of MPEO onto the backbone was confirmed using infra-red spectroscopy.

### Cosmetic Formulations

Polymers PG1-7 and PB 1-3 were used in cosmetic formulations to illustrate the invention.

### Formulation of Cosmetic Compositions

The ingredients used in manufacturing the compositions are listed in this section under manufacturer tradenames for convenience, together with the names issued to them INCI system. The commercially supplied materials used in this invention were of cosmetic or food grade.

Castor oil BP (Castor oil, INCI: ricinus communis oil) and Vanilla fragrance (INCI: partum) were supplied by William Hodgson & Co. Cerilla raffinee (Refined candelilla wax, INCI: Candelilla cera), Cerabeil bio-D (beeswax, INCI: Cera alba), Cerabeil White (beeswax, INCI: Cera alba), Cerauba T1 (prime yellow carnauba wax, INCI: Copemicia Cerifera [Carnauba] Wax), Cerozo D306 (Ozokerite Wax, INCI: Ozokerite) and Cerozo E626 (Ozokerite Wax, INCI: Ozokerite) were manufactured by Baerlocher. Microcrystalline wax (INCI: Microcrystalline wax) was manufactured by Pothe Hille & Co. Anhydrous Protalan Lanolin (INCI: Lanolin) and Vitamin E-USP (Vitamin E alcohol, INCI: DL-α-tocopheryl acetate) were supplied by Protameen Chemicals inc. Softisan 100P (saturated fatty acid glycerides, INCI: hydrogenated coco-glycerides), Mygliol 812N (fatty acid esters, INCI: caprylic/capric/triglycerides) and Softisan 645 (glycerol fatty acid ester, INCI: Bis-diglyceryl polyacyladipate-1) were manufactured by Sasol. Versagel M500 (gelled mineral oil, INCI: mineral oil (and) ethylene/propylene/styrene copolymer (and) butylene/ethylene/styrene copolymer) and Snow White Petrolatum USP (INCI: Petrolatum) was manufactured by Penreco. Dow Corning 5562 Carbino fluid (INCI: Bis-HydroxyethoxypropylDimethicone), Dow Corning 9701 Cosmetic Powder (INCI: Dimethicone/Vinyl Dimethicone Crosspolymer (and) Silica), Dow Corning 2-1184 FLUID (INCI: Dimethicone (and) Trisiloxane) and Dow Corning BY 11-030 (INCI: Cyclopentasiloxane (and) PEG/PPG-19/19 Dimethicone) were manufactured by Dow Corning. Microma 100 (INCI: Polymethyl methacrylate) was manufactured by the Ikeda Corporation. J-68-BC (INCI: Talc) was manufactured by the US Cosmetic Corporation. (INCI: Avocado oil unrefined (referred to henceforth as Avocado oil, INCI: persea gratissima, Blanova shea nut butter (referred to henceforth as Shea butter, INCI: Butyrospermum Parkii[Shea Butter]). LEXGARD O (INCI: Caprylyl Glycol) was manufactured by Inolex. Paraffin liquid (INCI: paraffinum liquidum) was manufactured by Merck. Deionised water (INCI:aqua) was obtained using standard laboratory techniques.

The pigments below were added for colour to illustrate the addition of colour to the cosmetic compositions by way of example. The invention is not limited to these colours and it will be understood by those skilled in the art that it will be possible to use another cosmetically acceptable colourant to change the colour to that desired.

Ronastar Noble Sparks (INCI: Calcium Aluminium Borosilicate, Silica, CI-77891 (Titanium Dioxide), Tin Oxide), Dichrona RY (INCI: Mica and Titanium dioxide (CI-77891) and Carmine (C175470), Ronaflair Mica M (INCI: Mica) and Colorona Carmine Red (Mica coated titanium dioxide and carmine, INCI: Mica and Titanium Dioxide and Carmine) were manufactured by Merck. COD 8002 (FD&C [food, drug and cosmetic] Red 6 dispersion in castor oil, INCI: Ricinus Communis and CI 15850 and BHT), COD 8006 (red Iron oxide dispersed in castor oil, INCI: Ricinus Communis and CI-77491 and BHT), COD 8007 (FD&C blue 1 lake castor oil dispersion, INCI: Ricinus Communis and CI-42090 and BHT) and COD 8008 (white titanium dioxide castor oil dispersion, INCI: Ricinus Communis and CI-77891 and BHT) were manufactured by Sun Chemical. SA-C47051-10 (INCI: CI-77891 and Dimethicone), SA-C331700-10 (INCI: CI-77492 and Dimethicone), SA-C332199-10 (INCI: CI-77491 and Dimethicone) and SA-C335000-10 (INCI: CI-77499 and Dimethicone) were manufactured by Miyoshi Kasei.

### Example 10

### Examination of the Compatibility of Some Amphiphilic Copolymers with Lip Product Ingredients

### (i) Thermal Properties of the Polymers

As lip product formulations are created by melting a mixture of the ingredients together and mixing them, the thermal properties of the polymers was tested. Samples of polymers (PG1-3 and PG6-7) were placed in an oven at 90° C. It was observed that after 30 minutes all of the polymers had become softer and PG2 had melted to form a viscous gel.

### (ii) Lipstick Base

PG1 was tested first in a basic formulation representing a lipstick base.

The ingredients in the table below were weighed out into in a beaker and heated to 105° C. for 1 hour. After this period the beaker was placed in a water bath maintained at at 95° C. and the mixture was mixed with a laboratory homogeniser (IKA Ultra-Turax) until a homogenous mixture was obtained. Formulations 10A and 10B were poured into a lipstick mould. The lipstick mould was manufactured by Kemwall Engineering and designed to manufacture a total of 20 sticks at a time. The internal surfaces of the lipstick mould were sprayed with Silicone oil (DC 200 fluid, Dow) to ensure the ease of removal of the sticks after manufacture. The formulation in the beaker was allowed to cool to 65° C. and poured into the lipstick mould which had been preheated to 40° C. The mould was allowed to cool to ambient temperature, then placed in a plastic bag and then into a fridge at 4° C. for 2 h. The sticks were then pushed into lipstick tubes. Formulation 10C was then placed into small cosmetic jars.

The replacement of the waxes in the formulation with PG1 led to a decrease of the strength of the lip products. Formulations 10A and 10B produced lipsticks. In the case of the highest amount of PG1 (33.3%) the resulting formulations were soft lip balms with a butter like texture. The organoleptic properties of the formulations were noticeably improved with the addition of PG1 however.

| | Formulation 10A | Formulation 10B | Formulation 10C |
|---|---|---|---|
| | Ingredient | Ingredient | Ingredient |
| Ingredient Name | Weight % | Weight % | Weight % |
| Polymer PG1 | 9.30 | 12.80 | 33.30 |
| Microcrystalline | 15.30 | 14.71 | 11.30 |
| wax | | | |
| Anhydrous | 24.00 | 23.07 | 0.00 |
| Protalan Lanolin | | | |
| Castor Oil BP | 21.20 | 20.38 | 25.20 |
| Paraffin Liquid | 30.00 | 28.84 | 30.00 |
| Vitamin E-USP | 0.20 | 0.20 | 0.20 |

### Example 11

### Examination of the Compatibility of Some Amphiphilic Block Copolymers with Lip Product Ingredients

A series of block copolymers were tested in lip product formulations. These materials were block copolymers of ethylene and ethylene oxide (Unithox 720, Polymer PB1) (UInithox 750, Polymer PB2), or propylene and ethylene oxide (X10044, Polymer PB3) manufactured by Baker Hughes.

The polymer PB1 was combined at a ratio of 2.5 and 5.0 with waxes and oils to make a solid cosmetic composition. The same methodology was used as in Example 10(ii) by adding the ingredients listed in Table one after the other. The formulations were poured into a lipstick mould at 75-80° C.

| | Formulation 11A | Formulation 11B |
|---|---|---|
| | Ingredient | Ingredient |
| Ingredient Name | Weight % | Weight % |
| Polymer PB1 | 5.00 | 2.50 |
| Cerozo D306 | 20.0 | 20.00 |
| Microcrystalline wax | 2.00 | 1.00 |
| Anhydrous Protalan | 23.00 | 15.00 |
| Lanolin | | |
| Castor Oil BP | 25.00 | 28.16 |
| Paraffin Liquid | 25.00 | 25.00 |
| COD 8008 | 0.00 | 1.16 |
| COD 8002 | 0.00 | 2.00 |
| COD 8007 | 0.00 | 0.23 |
| Colorona Carmine | 0.00 | 5.00 |
| red | | |
| Strength (g) | 571 | 445 |

The formulation containing 5 weight % PB1 (11A) did not contain any pigment and would be most suitable for a product marketed as a lip balm. The formulation with 5% of the polymer was harder than that with 2.5% polymer (11B) as might be expected from the addition of more of a material that is harder than the majority of the ingredients in the formulation.

Cosmetic compositions were also formulated using the same methodology and polymer PB2 as illustrated with the formulations in the table below:

| | Formulation 11C | Formulation 11D |
|---|---|---|
| | Ingredient | Ingredient |
| Ingredient Name | Weight % | Weight % |
| Polymer PB2 | 5.00 | 2.50 |
| Cerozo D306 | 20.00 | 20.00 |
| Microcrystalline wax | 2.00 | 1.00 |
| Anhydrous Protalan | 23.00 | 15.00 |
| Lanolin | | |
| Castor oil BP | 25.00 | 28.16 |
| Paraffin liquid | 25.00 | 25.00 |
| COD 8008 | 0.00 | 1.16 |
| COD 8002 | 0.00 | 2.00 |
| COD 8007 | 0.00 | 0.23 |
| Colorona Carmine | 0.00 | 5.00 |
| Red | | |
| Strength (g) | 594 | Not Measured |

The formulation without colour formed a lip balm product of adequate strength.

The same experiments were also repeated using amphiphilic copolymer PB3:

| | Formulation 11E | Formulation 11F |
|---|---|---|
| Ingredient Name | Ingredient | Ingredient |
| | Weight % | Weight % |
| Polymer PB3 | 5.00 | 2.50 |
| Cerozo D306 | 20.00 | 20.00 |
| Microcrystalline wax | 2.00 | 1.00 |
| Anhydrous Protalan | 23.00 | 15.00 |
| Lanolin | | |
| Castor Oil BP | 25.00 | 28.16 |
| Paraffin Liquid | 25.00 | 25.00 |
| COD 8008 | 0.00 | 1.16 |
| COD 8002 | 0.00 | 2.00 |
| COD 8007 | 0.00 | 0.23 |
| Colorona Carmine | 0.00 | 5.00 |
| Red | | |
| Strength (g) | 590 | 479 |

As would be predicted from the previous results the polymers formed sticks of reasonable hardness those with the greater amounts of polymer forming harder sticks due to its higher melting point compared with the rest of the ingredients. PB3 was found to have good compatibility with the pigment in castor oil it was tested with. This polymer is thus suitable for use in a range of lip products.

### Example 12

### Formulation of Polymer PG1 into Lip gloss

Using the methodology described in Example 10(ii) PG1 was mixed into a high oil lip gloss formulation. The final product was poured into small jars.

| | Standard | | |
|---|---|---|---|
| | Formulation 12A | Formulation 12B | Formulation 12C |
| Ingredient | Ingredient | Ingredient | Ingredient |
| Name | Weight % | Weight % | Weight % |
| Polymer PG1 | 0.00 | 2.44 | 6.80 |
| Castor Oil BP | 69.73 | 68.07 | 66.88 |
| Miglyol 808 | 20.00 | 19.52 | 18.64 |
| Cerabeil Bio | 2.50 | 2.44 | 1.70 |
| Cerauba T1 | 2.50 | 2.44 | 1.70 |
| Dichrona Ry | 5.00 | 4.88 | 4.10 |
| Vitamin E-USP | 0.20 | 0.19 | 0.19 |

The formulations were analysed by putting them on the lips of volunteers. The formulations containing PG1 were found to have superior organoleptic properties over those without the polymer. The products were perceived to be less oily, and have greater softness. In particular it was noted that they formed a film with enhanced adhesion, producing a longer lasting formulation.

### Example 13

### Formulation of Polymer PG1 into Hard Lip Balm

This formulation was prepared using the same methodology described in Example 10(ii) by adding the ingredients in the table below in the order listed:

| | | Standard |
|---|---|---|
| | Formulation | Formulation |
| | 13A | 13B |
| | Ingredient | Ingredient |
| Ingredient Name | Weight % | Weight % |
| Polymer PG1 | 10.45 | 0.00 |
| Cerilla Raffine | 7.46 | 8.33 |
| Microcrystalline Wax | 2.98 | 3.33 |
| Avocado Oil Unrefined | 15.00 | 16.67 |
| Castor Oil BP | 59.50 | 66.50 |
| Blanova Shea Nut | 4.26 | 4.76 |
| Butter | | |
| Vitamin E-USP | 0.18 | 0.20 |
| Vanilla Fragrance | 0.18 | 0.20 |

The resulting product was tested on the lips and back of the wrist of volunteers and found to be suitable for use as a hard lip balm product. The addition of polymer PG1 gave enhanced organoleptic properties and perception of adhesion relative to that without (13B)

### Example 14

### Formulation of Polymer PG1 into Soft Lip Balm

This formulation was prepared using the same methodology described in Example 10(ii) and the ingredients in the table below.

| | Standard | |
|---|---|---|
| | Formulation 14A | Formulation 14B |
| | Ingredient | Ingredient |
| Ingredient Name | Weight % | Weight % |
| Polymer PG1 | 0.00 | 19.00 |
| Cerabeil Bio D | 3.00 | 2.43 |
| Softisan 645 | 15.00 | 12.16 |
| Cerilla Raffine | 4.50 | 3.65 |
| Cerauba T1 | 1.00 | 0.81 |
| Miglyol 812 N | 75.80 | 61.46 |
| Vitamin E-USP | 0.20 | 0.16 |
| Vanilla Fragrance | 0.50 | 0.40 |

The addition of polymer PG1 was found to give a pronounced improvement on the organoleptic properties of the formulation. The resulting product was a soft lip balm with a creamy and pleasant texture. Despite the presence of an oil with an adhesive benefit (Softisan 645) in the standard 14A, the complete replacement of Softisan 645 with PG1 led to a product with an increase in adhesion relative to the standard 14A.

### Example 15

### Formulation of Polymer PG1 into Lipstick and Study Via Panel Tests

The lipstick formulations were prepared using the same methodology described in Example 10(ii) and the ingredients in the table below.

| | Formulation 15A | Formulation 15B |
|---|---|---|
| | Ingredient | Ingredient |
| Ingredient Name | Weight % | Weight % |
| Polymer PG1 | 0.00 | 10.00 |
| Cerozo D306 | 19.5 | 17.6 |
| Microcrystalline Wax | 1.11 | 1.00 |
| Anhydrous Protalan Lanolin | 18.22 | 20.00 |
| Castor Oil BP | 23.00 | 20.00 |
| Paraffin Liquid | 29.00 | 27.40 |
| COD 8008 | 2.22 | 2.00 |
| COD 8006 | 0.66 | 0.60 |
| Colorona Carmine Red | 3.50 | 3.50 |
| Vitamin E-USP | 0.20 | 0.20 |
| Vanilla Fragrance | 0.20 | 0.20 |

The lip balm formulations were prepared using the same methodology described in Example 10(ii) and the ingredients in the table below.

| | Formulation 15C | Formulation 15D |
|---|---|---|
| | Ingredient | Ingredient |
| Ingredient Name | Weight % | Weight % |
| Polymer PG1 | 0.00 | 9.87 |
| Cerozo D306 | 21.37 | 18.26 |
| Microcrystalline Wax | 1.21 | 1.09 |
| Anhydrous Protalan Lanolin | 19.97 | 18.00 |
| Castor Oil BP | 25.21 | 22.72 |
| Paraffin Liquid | 31.78 | 28.64 |
| Vitamin E-USP | 0.20 | 0.20 |
| Vanilla Fragrance | 0.20 | 0.20 |

The lip balms were poured into small jars whilst the formulations were still warm.

### Panel Tests

In order to assess the key properties of lip product formulations namely their feel and cosmetic appearance it is desirable to measure their sensorial properties directly on human skin. This can be achieved by applying the product directly to the arm for instance as a quick measure of their suitability and desirability. The manufactured lip products were tested by applying the product to the back of the wrist, tests of lipstick, lip gloss and lip balm formulations containing the amphiphilic copolymers revealed that the products formed cosmetically desirable formulations. Some of the products were more thoroughly tested on the lips of human volunteers to compare the effect of adding the amphiphilic copolymer to them.

The trial was performed as a blind trial, with the samples labeled randomly so the testers and sample distributors were unaware of the composition of the samples they were testing. The testers were required to sequentially test each formulation and answer a series of questions regarding the samples. The testers were asked to apply the stick at least twice a day or more if they desired to. The first series of questions allowed to the panel to describe their general feeling regarding some of the properties. And the second part of this test allowed the panel to score the properties from 0 to 10 by comparison. A description of the properties that the lip product was rated by are outlined below.

Spreadability: defined as the ease with which it is possible to get a uniform application of the lipstick; this has some relation to the amount of material imparted on the lips during application (also described as the pay-off or deposit).

Softness: the tendency of the product to feel soft on first application. Products with low scores will feel hard and dry when being applied.

Creaminess: at the moment of application and during wear a lip product may be perceived as creamy dry, or oily on the lips.

Shine/Gloss: the tendency of the product to reflect light when on the lips in an aesthetically pleasing manner.

Moisturisation: this is the tendency for a product to leave lips with a greater amount of moisture or the perception of more moisturisation. The panel tests measure the tester's perceived change in moisture levels.

Adhesiveness: the perceived strength of the interaction between the product and the wearer's lips. Adhesiveness is necessary to increase the transfer resistance of the product, that is the resistance of the product to move from the lips to a secondary substrate they come in contact with. This property is important in increasing the length of time that a product stays on the wearer's lips, in other words creating a longer lasting formulation.

In addition the testers were asked for other comments including which formulation they preferred.

### Results:

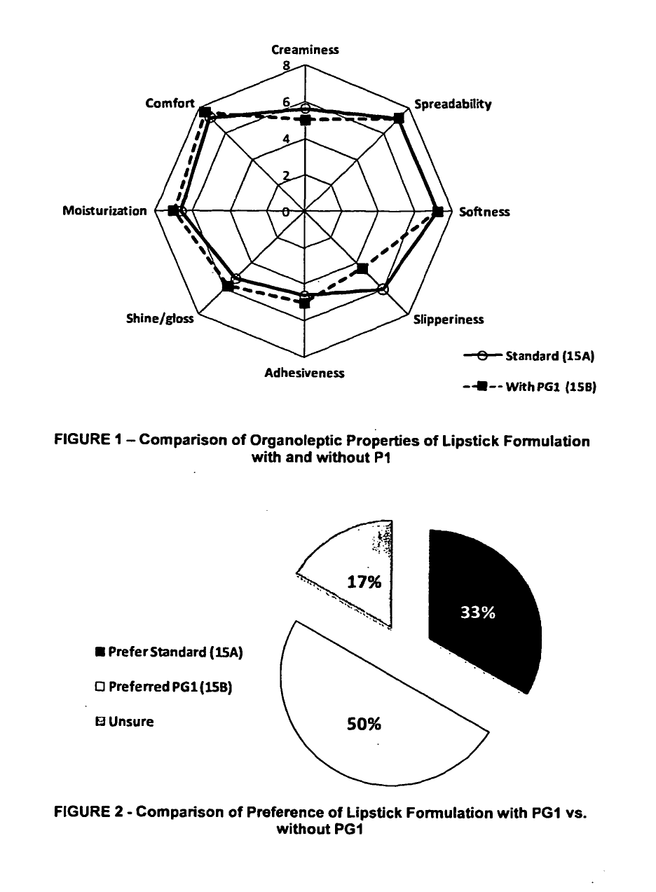

The data was collated and is depicted in the form of a radar (or spider) chart (FIG. 1). In most aspects the formulation with PG1 was superior or equal to that of the standard. A slight increase in both moisturisation and adhesiveness were noted, the later correlating with the perception that the formulation stayed longer on the lips compared with the standard.

It was clear that the lipstick formulation containing PG1 was also preferred by the greatest majority of testers (FIG. 2) with the majority of respondents preferring the PG1 containing formulation (50%) compared with standard (33%).

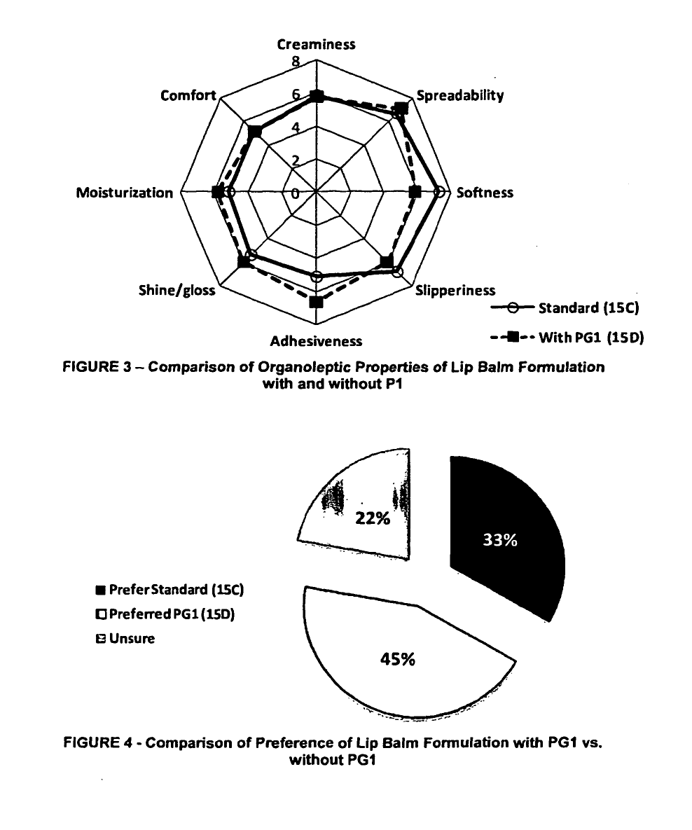

A similar case was observed in the case of the comparison of the organoleptic properties of the lip balm with (formulation 15D) and without (15C) PG1. In most cases the properties were either similar or superior in the case of the formulation with PG1 and a notable increase in adhesion is observed in the data (FIG. 3).

As with the lipstick the largest portion (45%) of the testers preferred the formulation with PG1 (FIG. 4).

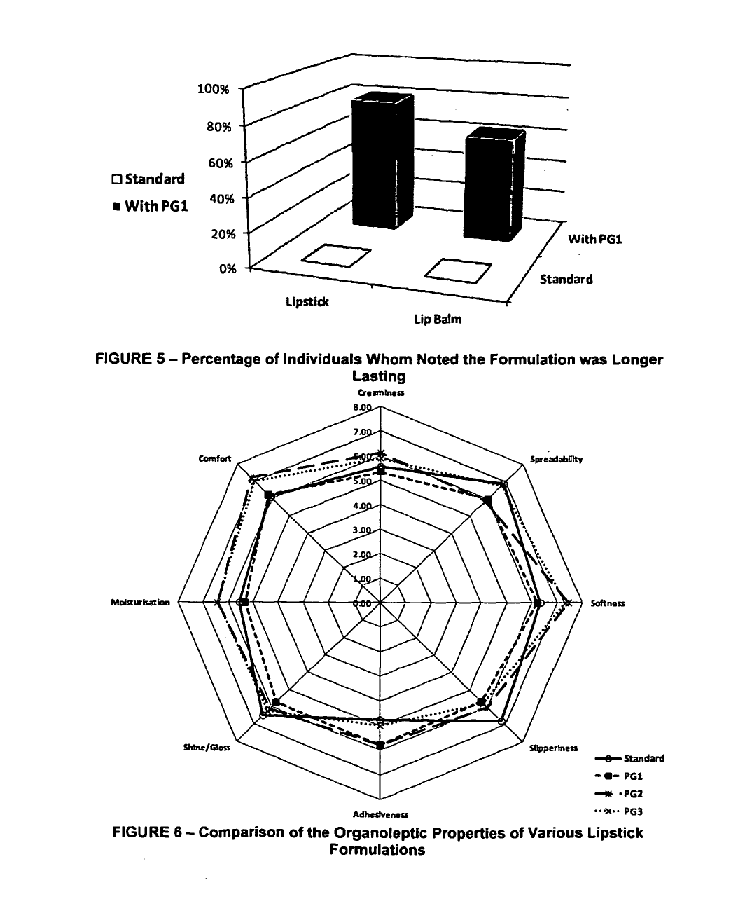

Individuals testing both the lipstick and lip balm formulations were asked what the most notable property of the formulation was. In the case of both the formulations the majority of the individuals noted the formulation containing PG1 was longer lasting (FIG. 5) compared with no individuals in the case of the standard.

### Example 16

### Formulation of Polymers PG1. PG2 and PG3 into Lipstick

This formulation was prepared using the same methodology described in Example 10(ii) and the ingredients in the table below.

| | Standard | | | |
|---|---|---|---|---|
| | Formulation | Formulation | Formulation | Formulation |
| | 16A | 16B | 16C | 16D |
| | Ingredient | Ingredient | Ingredient | Ingredient |
| Ingredient Name | Weight % | Weight % | Weight % | Weight % |
| Polymer PG1 | 0.00 | 10.00 | 0.00 | 0.00 |
| Polymer PG2 | 0.00 | 0.00 | 10.00 | 0.00 |
| Polymer PG3 | 0.00 | 0.00 | 0.00 | 10.00 |
| Ozokerite Wax | 19.50 | 17.72 | 17.72 | 17.72 |
| Microcrystalline | 1.11 | 1.00 | 1.00 | 1.00 |
| Wax | | | | |
| Anhydrous | 18.22 | 16.56 | 16.56 | 16.56 |
| Protalan | | | | |
| Lanolin | | | | |
| Castor Oil BP | 23.00 | 20.90 | 20.90 | 20.90 |
| Paraffin Oil | 29.00 | 26.36 | 26.36 | 26.36 |
| COD 8008 | 1.15 | 1.04 | 1.04 | 1.04 |
| COD 8002 | 2.00 | 1.81 | 1.81 | 1.81 |
| COD 8007 | 0.20 | 0.18 | 0.18 | 0.18 |
| Colorona | 5.00 | 4.54 | 4.54 | 4.54 |
| Carmine Red | | | | |
| Ronastar Noble | 0.50 | 0.50 | 0.50 | 0.50 |
| Sparks | | | | |
| Vanilla Fragrance | 0.20 | 0.20 | 0.20 | 0.20 |
| Vitamin E-USP | 0.20 | 0.20 | 0.20 | 0.20 |

The formulations were tested using the panel test methodology described in Example 15. The results are plotted on a radar chart (FIG. 6) to help illustrate the invention. As will be apparent from the chart subtle changes to the nature of the polymer allow optimization of the organoleptic properties of the cosmetic compositions. In most respects the formulations outperform or equal that of the standard. In particular the enhanced adhesiveness of PG1 and PG2 compared with the standard is visible. Most participants noted that all three formulations containing amphiphilic graft copolymers were longer lasting than that without (formulation 16A).

### Example 17

### Formulation of Polymers PG2 and PG3 into Lip Gloss

This formulation was prepared using the same methodology described in Example 10(ii) and the ingredients in the table below.

| | Standard | | |
|---|---|---|---|
| | Formulation | Formulation | Formulation |
| | 17A | 17B | 17C |
| | Ingredient | Ingredient | Ingredient |
| Ingredient Name | Weight % | Weight % | Weight % |
| Polymer PG2 | 0.00 | 19.00 | 0.00 |
| Polymer PG3 | 0.00 | 0.00 | 19.00 |
| Cerabeil Bio D | 3.00 | 2.43 | 2.43 |
| Softisan 645 | 15.00 | 12.16 | 12.16 |
| Cerilla Raffine | 4.50 | 3.65 | 3.65 |
| Cerauba T1 | 1.00 | 0.81 | 0.81 |
| Miglyol 812 N | 75.80 | 61.46 | 61.46 |
| Vitamin E-USP | 0.20 | 0.16 | 0.16 |
| Vanilla Fragrance | 0.50 | 0.40 | 0.40 |

The formulations were poured into jars whilst still warm.

### Example 18

### Formulation of Polymer PG4 into Lip Gloss

Using the methodology described in Example 10(ii) polymer PG4 was mixed into a high oil lip gloss formulation.

| | Formulation 18A |
|---|---|
| | Ingredient |
| Ingredient Name | Weight % |
| Polymer PG4 | 6.80 |
| Castor Oil BP | 66.88 |
| Miglyol 808 | 18.64 |
| Cerabeil Bio | 1.70 |
| Cerauba T1 | 1.70 |
| Dichrona Ry | 4.10 |
| Vitamin E-USP | 0.19 |

The final product was poured into small jars. The resulting product was found to be a homogenous cosmetic composition in the form of lip gloss with the benefit of the presence of polymer PG4.

### Example 19

### Formulation of Polymer PG5 into Lip Gloss

Using the methodology described in Example 10(ii) polymer PG5 was mixed into a high oil lip gloss formulation.

| | Formulation 19A |
|---|---|
| | Ingredient |
| Ingredient Name | Weight % |
| Polymer PG5 | 6.80 |
| Castor Oil BP | 66.88 |
| Miglyol 808 | 18.64 |
| Cerabeil Bio | 1.70 |
| Cerauba T1 | 1.70 |
| Dichrona Ry | 4.10 |
| Vdamin E-USP | 0.19 |

The final product was poured into small jars. The resulting product was found to be a homogenous cosmetic composition in the form of lip gloss with the benefit of the presence of polymer PG5.

### Example 20

### Formulation of Polymers PG6 and PG7

This formulation was prepared using the same methodology described in Example 10(ii) and the ingredients in the table below:

| | Formulation 20A | Formulation 20B |
|---|---|---|
| | Ingredient | Ingredient |
| Ingredient Name | Weight % | Weight % |
| Polymer PG6 | 5.00 | 0.00 |
| Polymer PG7 | 0.00 | 5.00 |
| Cerozo D306 | 20.00 | 20.00 |
| Microcrystalline Wax | 2.00 | 2.00 |
| Anhydrous Protalan Lanolin | 23.00 | 23.00 |
| Castor Oil BP | 25.00 | 25.00 |
| Paraffin Liquid | 25.00 | 25.00 |

The two formulations that were produced may be regarded as suitable for use as a lip balm.

Polymers PG6 and PG7 were compared by placing them into a lipstick formulation using the methodology outlined in a similar methodology to that outlined in Example 10(ii):

| | Formulation 20C | Formulation 20D |
|---|---|---|
| | Ingredient | Ingredient |
| Ingredient Name | Weight % | Weight % |
| Polymer PG6 | 5.00 | 0.00 |
| Polymer PG7 | 0.00 | 5.00 |
| Cerozo D306 | 20.00 | 20.00 |
| Microcrystalline Wax | 1.00 | 1.00 |
| Anhydrous Protalan Lanolin | 15.00 | 15.00 |
| Castor Oil BP | 28.16 | 28.16 |
| Paraffin Liquid | 25.00 | 25.00 |
| COD 8008 | 1.16 | 1.16 |
| COD 8002 | 2.00 | 2.00 |
| COD 8007 | 0.23 | 0.23 |
| Colorona Carmine Red | 5.00 | 5.00 |

PG7 was found to produce a particularly homogenous lipstick formulation. The hydrophilic polymer PG6 is particularly suitable for cosmetic compositions that do not contain pigment, like lip balm.

### Example 21

### Formulation of Polymer PG1 into Compact Foundation

This formulation is for a foundation designed for application as face make-up. It was assembled using the formulation below (Dow Corning is abbreviated as DC) and the method described below.

| | | Standard | |
|---|---|---|---|
| | | Formulation 21A | Formulation 21B |
| | | Ingredient | Ingredient |
| Phase | Ingredient Name | Weight % | Weight % |
| A | DC 9701 Cosmetic Powder | 5.00 | 5.00 |
| | Microma 100 | 4.00 | 4.00 |
| | SA-C47051-10 | 8.00 | 8.00 |
| | SA-C331700-10 | 1.50 | 1.50 |
| | SA-C332199-10 | 0.35 | 0.35 |
| | SA-C335000-10 | 0.10 | 0.10 |
| | Ronaflair Mica M | 2.05 | 2.05 |
| | J-68-BC | 10.00 | 10.00 |
| B | DC 5562 Carbino Fluid | 16.80 | 16.80 |
| | Phytosqualane | 10.00 | 10.00 |
| | Snow White Petrolatum USP | 2.00 | 2.00 |
| C | Cerozo E 626 | 6.00 | 6.00 |
| | Cerabeil White | 3.00 | 0.00 |
| | Polymer PG1 | 0.00 | 3.00 |
| | DC 2-1184 Fluid | 25.00 | 25.00 |
| | DC BY 11-030 | 1.00 | 1.00 |
| D | Lexgard O | 0.09 | 0.09 |
| | Deionised Water | 5.00 | 5.00 |

The ingredients listed in phase A were weighed into a beaker and the combined mixture placed in a grinder designed for use with coffee beans and food. The ingredients were then ground into a homogenous mixture, it will be appreciated by those skilled in the art that any mixture suitable for the mixture of pigments would be suitable.

Phase D was prepared by dissolving the Lexgard O in the water with gentle heating.

The ingredients in phase B were weighed into a beaker and subsequently placed into an oil bath maintained at 50° C. and mixed with an overhead stirrer. When the mixture was homogenous, phase A was added slowly with continued mixing. The temperature of the water bath was then increased to 85° C. and stirring discontinued.

The waxes in phase C were weighed out and mixed into the formulation. When the waxes had fully melted stirring recommenced and continued until the mixture was homogenous, whilst the temperature of the mixture was allowed to fall to 60° C. Phase D was then added slowly to the formulation and the cosmetic mixed thoroughly until the formulation was homogenous. It was then poured into appropriate packaging.

After rub down on the skin the formulation with polymer PG1 (formulation 21B) was found to possess similar properties to that without PG1, with the further benefit of the feeling of enhanced adhesiveness.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

1. A cosmetic composition comprising:
(i) at least one amphiphilic copolymer; and
(ii) one or more cosmetically acceptable diluents, excipients or carriers;
wherein the amphiphilic copolymer is selected from the group consisting of a graft copolymer comprising a hydrophobic straight or branched chain carbon-carbon backbone having at least one hydrophilic side chain attached thereto; a graft copolymer comprising a hydrophilic straight or branched chain backbone having at least one hydrophobic side chain attached thereto; a block copolymer comprising at least one hydrophilic block and at least one hydrophobic block in a straight or branched chain backbone; and a cross-linked/network copolymer.

2. A cosmetic composition according to claim 1, wherein the amphiphilic copolymer is a graft copolymer comprising a hydrophobic straight or branched chain carbon-carbon backbone having at least one hydrophilic side chain attached thereto.

3. A cosmetic composition according to claim 2, wherein the hydrophilic side chains are each independently of formula (I), 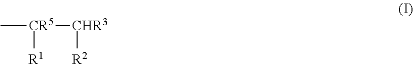
wherein R¹ and R² are each independently H₁—C(O)WR⁴ or —C(O)Q;
provided that at least one of R¹ and R² is the group —C(O)Q;
or R¹ and R² together form a cyclic structure together with the carbon atoms to which they are attached, of formula (II) 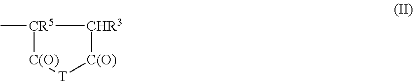
wherein:
R³ and R⁵ are each independently H or alkyl;
W is O or NR⁴;
Q is a group of formula —X¹—Y—X²P;
T is a group of formula —N—Y—X²—P;
X¹ is O, S or NR⁴;
X² is O, S, (CH₂)ₚ or NR⁴;
p is 0 to 6;
each R⁴ is independently H or alkyl;
P is H or another backbone; and
Y is a hydrophilic polymeric group.

4. A cosmetic composition according to claim 2, wherein the graft copolymer has from about 1 to about pendant hydrophilic side chains attached thereto.

5. A cosmetic composition according to claim 3, wherein the hydrophilic polymeric group Y is a polyalkylene oxide, polyglycidol, poly(vinyl alcohol), polyethylene imine), poly(styrene sulphonate) or poly(acrylie acid).

6. A cosmetic composition according to claim 3, wherein the hydrophilic polymeric group Y is of formula -(Alk¹-O)_{b}-(Alk²-O)_{c}, wherein Alk¹ and Alk² are each independently an alkylene group having from 2 to 4 carbon atoms, and b and c are each independently an integer from 1 to 125; provided that the sum of b+c has a value in the range of from about 10 to about 600.

7. A cosmetic composition according to claim 1, wherein the carbon-carbon backbone is derived from a homopolymer of an ethylenically-unsaturated polymerizable hydrocarbon monomer or from a copolymer of two or more ethylenically-unsaturated polymerizable hydrocarbon monomers.

8. A cosmetic composition according to claim 7, wherein the carbon-carbon backbone is derived from ethylene, isobutylene, 1,3-butadiene, isoprene, a C10-C20 terminal alkene, such as octadecene, styrene, or a mixture thereof.

9. A composition according to claim 7, wherein the carbon-carbon backbone has maleic anhydride, maleic acid or salts thereof or maleic acid ester or salts thereof or a mixture thereof pendant thereto.

10. A cosmetic composition according to claim 9, wherein the carbon-carbon backbone comprises from about 1 to about 50 mol % maleic anhydride

11. A cosmetic composition according to claim 1, wherein the carbon-carbon backbone is a copolymer of:
(i) maleic anhydride, maleic acid or salts thereof or maleic acid ester or salts thereof or a mixture thereof; and
(ii) ethylenically-unsaturated polymerizable monomer or from a copolymer of two or more ethylenically-unsaturated polymerizable monomers.

12. The composition according to claim 11 wherein the ethylenically-unsaturated polymerizable monomer is ethylene, isobutylene, 1,3-butadiene, isoprene, a C10-C20 terminal alkene, such as octadecene, styrene, or a mixture thereof.

13. A composition according to claim 11, wherein the amphiphilic copolymer backbone is an alternating copolymer of maleic anhydride, maleic acid or salts thereof or maleic acid ester or salts thereof and the ethylenically-unsaturated polymerizable monomer.

14. A cosmetic composition according to claim 1, wherein the amphiphilic copolymer is prepared by reacting a compound of formulae (III), (IX) or (X), 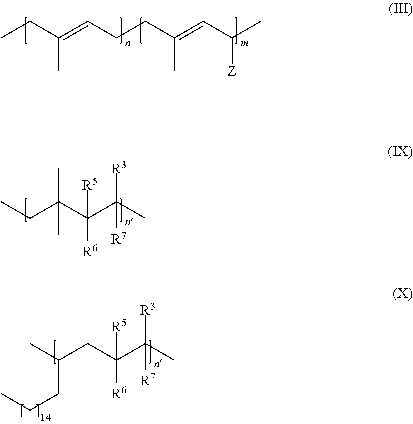
wherein Z is a group of the formula (IV), 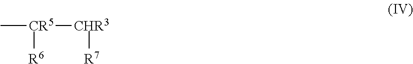
wherein R³ and R⁵ are each independently H or alkyl, and R⁶ and R⁷ are each independently H or an acyl group, provided that at least one of R⁶ and R⁷ is an acyl group, or R⁶ and R⁷ are linked to form, together with the carbon atoms to which they are attached, a group of formula (V), 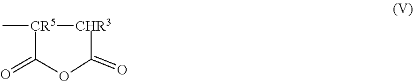
where n and m are each independently an integer from 1 to 20000 and n′ is an integer from 5 to 4000;
with a side chain precursor of formula (VI)
HX¹—Y—X²P (VI)
wherein:
X¹ is O, S or NR⁴;
X² is O, S, (CH₂)ₚ or NR⁴;
p is 0 to 6;
each R⁴ is independently H or alkyl;
P is H or another backbone; and
Y is a hydrophilic polymeric group.

15. A cosmetic composition according to claim 14, wherein the amphiphilic copolymer is prepared by reacting a compound of formulae (IIIa), (IXa) or (Xa), 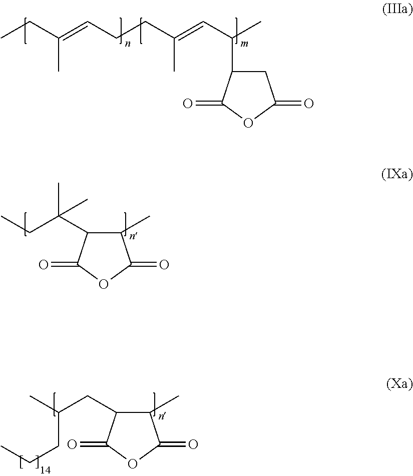
where n and m are each independently an integer from 1 to 20000 and n′ is an integer from 5 to 4000, with the side chain precursor of formula (VI).

16. A cosmetic composition according to claim 14, wherein the amphiphilic copolymer is prepared by reacting a polymer precursor of formulae (IIIb), (IXb) or (Xb), 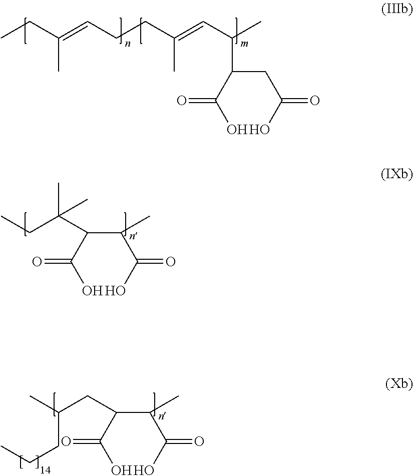
where n and m are each independently an integer from 1 to 20000 and d is an integer from 5 to 4000, with the side chain precursor of formula (VI).

17. A cosmetic composition according to claim 14, wherein the amphiphilic copolymer is prepared by reacting a polymer precursor of formulae (IIIc), (IXc) or (Xc), 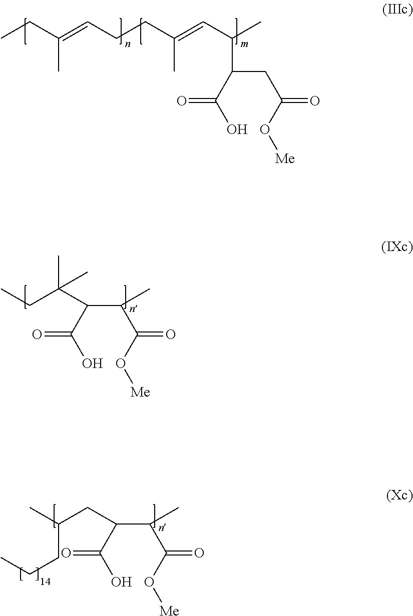
where n and m are each independently an integer from 1 to 20000 and n′ is an integer from 5 to 4000, with the side chain precursor of formula (VI).

18. A cosmetic composition according to claim 14, wherein said side chain precursor is of formula (VIa) 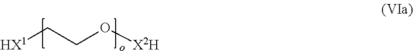
wherein X¹ is O or NH and X² is (CH₂)ₚ and o is an integer from 5 to 600.

19. A cosmetic composition according to claim 14, wherein said side chain precursor is of formula (VIb) 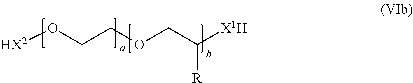
wherein R is H or alkyl, X¹ is O or NH and X² is CH₂ and the sum of a and b is an integer from 5 to 600.

20. A cosmetic composition according to claim 19, wherein said side chain precursor is of formula (VIc) 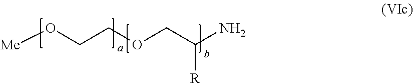
wherein R is H alkyl and the sum of a and b is an integer from 5 to 600.

21. A cosmetic composition according to claim 1, wherein the amphiphilic copolymer is of formula (VII): 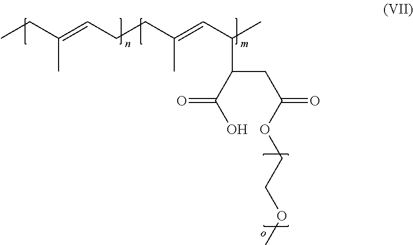
wherein each of m and n is independently an integer from 1 to 20000 and o is an integer from 5 to 600.

22. A cosmetic composition according to claim 1, wherein the amphiphilic copolymer is of formula (VIII): 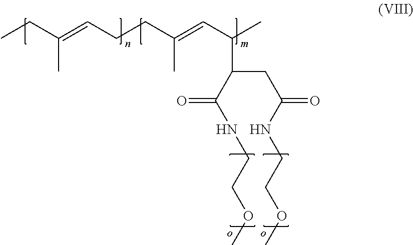
wherein each of m and n is independently an integer from 1 to 20000 and o is an integer from 5 to 600.

23. A cosmetic composition according to claim 1, wherein the amphiphilic copolymer is a block comprising hydrophilic blocks and hydrophobic blocks in a straight or branched chain backbone.

24. A cosmetic composition according to claim 23, wherein the straight or branched chain backbone has at least one side chain attached thereto.

25. A cosmetic composition according to claim 23, wherein the block copolymer has the structure: 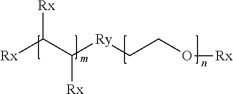
wherein Rx is an alkyl, aryl or H and Ry is O, NRₓSi(Rx)₂.

26. A cosmetic composition according to claim 23, wherein the block copolymer is a copolymer of ethylene oxide and an alkene, diene or polyene.

27. A cosmetic composition according to claim 26, wherein the block copolymer is a copolymer of ethylene oxide and ethylene, propylene, isoprene or butadiene.

28. A cosmetic composition according to claim 23, wherein the block copolymer has the structure: 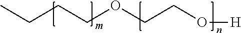
wherein m is 3 to 100 n is independently 3 to 100.

29. A cosmetic composition according to claim 23, wherein the block polymer has the structure: 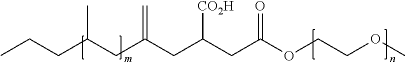
wherein m is 3 to 100 n is 3 to 100.

30. A cosmetic composition according to claim 1, wherein the amphiphilic copolymer is a graft copolymer comprising a hydrophilic straight or branched chain backbone having at least one hydrophobic side chain attached thereto.

31. A cosmetic composition according to claim 30, wherein the hydrophilic straight or branched chain backbone is a poly(alkylene oxide), polyglycidol, poly(vinyl alcohol), poly(ethylene imine), poly(styrene sulphonate) or poly(acrylic acid).

32. A cosmetic composition according to claim 20, wherein the hydrophobic side chain is a hydrocarbon.

33. A cosmetic composition according to claim 32, wherein the hydrophobic side chain is derived from ethylene, isobutylene, 1,3-butadiene, isoprene, a C10-C20 terminal alkene, such as octadecene, styrene, or a mixture thereof.

34. A cosmetic composition according to claim 30, wherein the graft copolymer has from about 1 to about pendant hydrophobic side chains attached thereto.

35. A cosmetic composition according to claim 1, wherein the amphiphilic copolymer is a cross-linked/network copolymer.

36. A cosmetic composition according to claim 1, wherein the cosmetic composition is selected from the group consisting of lip care and cosmetic products, face cosmetic and make-up products, and eye make-up products.

37. A cosmetic composition according to claim 1, wherein the cosmetically acceptable diluent, excipient or carrier is selected from an oil, a fat and a wax, or mixtures thereof.

38. A cosmetic composition according to claim 1, which further comprises one or more of an emollient, a colorant, a moisturizer, a UV blocker, an active agent, an antioxidant, a vitamin, a fragrance, a flavourant, a sweetener, a vegetable or herb extract and/or a preservative.

39. A cosmetic composition according to claim 36, which is a lip care or cosmetic product.

40. A cosmetic composition according to claim 39 further comprising a lip plumping agent.

41. A cosmetic composition according to claim 36, which is a face cosmetic or make-up product.

42. A cosmetic composition according to claim 36, which is an eye cosmetic product.

43. A process for the cosmetic composition according to claim 1, said process comprising melting and mixing the amphiphilic copolymer with the one or more cosmetically acceptable diluents, excipients or carriers to form a homogenous product.
